(19) United States
(12) Patent Application Publication
(10) Pub. No.: US 2026/0363891 A1
(43) Pub. Date: Nov. 19, 2026

(54) HINGED KNEE BRACE

(71) Applicant: YEN-TING LIAO, Taichung City (TW)

(72) Inventor: SU-CHANG LIAO, Taichung City (TW)

(21) Appl. No.: 19/644,294

(22) Filed: Apr. 10, 2026

(30) Foreign Application Priority Data
May 13, 2025 (TW) 114117875

(51) Int. Cl. A61F 5/01

(52) U.S. Cl. CPC A61F 5/0125; A61F 5/012; A61F 2005/0172

(57) ABSTRACT

A hinged knee brace includes a knee booster, a thigh support, and a calf support. The knee booster includes a bladder body having a pair of lateral sides, an actuating end portion, a limiting end portion, and at least one bladder chamber. The outer periphery of the lateral sides has deformable portions cooperating with elbow hinges of an elbow hinge assembly. An actuating link and a limiting link are disposed on and cooperate with the actuating end portion and the limiting end portion. The thigh support includes a main arm body, a connecting portion, and a first fixing seat that is connected to the actuating link and has a first pivot seat. The calf support includes a limiting arm, a support portion, and a second fixing seat that is connected to the limiting link and has a second pivot seat pivotally connected to the first pivot seat through a pivot.

### FIELD OF THE INVENTION

The present invention relates to a knee brace, and more particularly to a hinged knee brace for rehabilitation or training therapy for patients with knee joint injuries, providing flexible limitation for the movement of the knee joint when the user walks, cushioning support during squatting, and assistance in standing up after squatting.

### BACKGROUND OF THE INVENTION

The knee joint is a complex structure that bears the body’s weight and enables us to walk and jump. It is ingeniously composed of sturdy bones, flexible cartilage, cushioning menisci, and a series of ligaments and tendons. Due to its importance and frequent use in daily activities, it is one of the joints most prone to injury. There are many common types of knee joint injuries, including anterior cruciate ligament (ACL) injuries, meniscus injuries, chondromalacia patellae, iliotibial band syndrome, and knee osteoarthritis. Most of these injuries are caused by improper or excessive exercise and are primarily treated through rehabilitation, physical therapy, strength training, or injection therapy; in some cases, surgery may even be necessary. Knee osteoarthritis (degenerative knee arthritis) is one of the most common types of knee injuries. It is a degenerative process characterized by the gradual wear and tear of cartilage, caused by aging, excessive body weight, or overuse of the knee joint, and its treatment and recovery period are relatively long. During rehabilitation and recovery following treatment or surgery for a knee injury, a knee brace is often used to protect the knee joint. If the ligaments have previously experienced significant tears resulting in loss of joint capsule function, or if the reconstructed ligament tissue functions poorly and the joint cannot be stabilized solely by strengthening the muscles, or if the patient is still in the transitional rehabilitation period after injury without completing full training, a supportive knee brace (or knee support) may be used to provide additional support, assist in stabilizing the knee joint, and reduce excessive stress on the myofascial tissues.

Conventional supportive knee braces (or knee supports) mainly consist of an angle limiting disc pivotally connected between upper and lower supports made of a rigid material (such as metal). After the upper and lower supports are bound (secured with straps) to the thigh and calf, the limiting disc restricts the movement of the knee joint. While the knee brace provides support and restricts the movement of the knee joint, long-term reliance on the knee brace will reduce opportunities to exercise the muscles (posing a risk of muscle atrophy). Even though the knee brace allows for adjustment of the range of motion, enabling patients to perform squatting or sitting exercises, the user still needs to use an assistive device to pull him/her up when standing from a squatting position. This may somewhat affect the patient’s quality of life (for example, when it is inconvenient to carry an assistive device while going out or when no assistive device is available). If this type of brace provides a cushioning support when the patient squats and assists in standing up from a squatting position, and if both the cushioning support and the assistive force are adjustable, the patient will be able to recover the knee joint to a healthy state as quickly as possible while maintaining a good quality of life, in cooperation with rehabilitation and training therapy.

### SUMMARY OF THE INVENTION

The primary object of the present invention is to provide a hinged knee brace, comprising a knee booster, a thigh support, and a calf support.

The knee booster includes a fan-shaped bladder body. The bladder body has a pair of lateral sides, an actuating end portion and a limiting end portion at two ends of the lateral sides, and at least one bladder chamber inside the bladder body. The bladder body is formed of an airtight material. The bladder body is in the form of an expandable, deformable flexible bladder. Peripheries of the lateral sides, between the actuating end portion and the limiting end portion, are defined as an outer periphery and an inner periphery that share a common center point. The outer periphery is formed with a continuous corrugated configuration, thereby forming a plurality of deformable portions. The inner periphery is formed with folds, thereby forming a folded surface that allows for unfolding and folding. An elbow hinge assembly is disposed on the actuating end portion, the limiting end portion, and the plurality of deformable portions of the outer periphery. The elbow hinge assembly includes a plurality of elbow hinges that are pivotally connected to one another and correspond in number to the plurality of deformable portions. An actuating link and a limiting link are pivotally connected to two ends of the connected elbow hinges. Each of the plurality of elbow hinges includes a first hinge element and a second hinge element that are pivotally connected to each other and have supporting rigidity and cooperates with a corresponding one of the deformable portions. The actuating link is pivotally connected to one end of the connected elbow hinges and fitted onto the actuating end portion and has supporting rigidity. The limiting link is pivotally connected to the other end of the connected elbow hinges and fitted onto the limiting end portion and has supporting rigidity. The bladder body has at least one connector. The at least one connector has a passage therein. The passage is in fluid communication with the at least one bladder chamber, enabling a fluid in the at least one bladder chamber to enter and exit through the at least one connector.

The thigh support is an arm-shaped frame having supporting rigidity. The thigh support includes a main arm body, a connecting portion, and a first fixing seat. The main arm body is configured for being secured to a user’s thigh with a fastening strap. The connecting portion extends from one side of a lower end of the main arm body. The first fixing seat is formed on the connecting portion. The first fixing seat is coupled with the actuating end portion of the bladder body and connected to the actuating link. A first pivot seat, centered on the center point, is formed on one side of the first fixing seat.

The calf support is in the form of an arm body with tensile strength. The calf support includes a limiting arm, a support portion, and a second fixing seat. The support portion extends from a lower end of the limiting arm. The second fixing seat is disposed on an upper end of the limiting arm. The second fixing seat is coupled with the limiting end portion of the bladder body and connected to the limiting link. A second pivot seat corresponding to the first pivot seat is formed on one side of the second fixing seat. The second pivot seat is pivotally connected to the first pivot seat through a pivot.

When a fluid is supplied from a fluid supply system to the at least one bladder chamber of the knee booster via at least one connector, the space inside the at least one bladder chamber expands, enabling the plurality of deformable portions of the bladder body to expand and deform. Thus, the angle between the first hinge element and the second hinge element of each of the plurality of elbow hinges increases, generating an accumulated thrust that is concentrated on the actuating link, such that the main arm body of the thigh support, pivoted about the pivot, bears the thrust from the actuating link and performs an outward swing motion. The action of transmitting the thrust generated by the knee booster to the main arm body is defined as a boosting action of the knee booster. Conversely, when an external force is applied to the main arm body, causing the main arm body to perform an inward swing motion, the actuating link is driven to push the plurality of elbow hinges so that the angle between the first hinge element and the second hinge element of each of the plurality of elbow hinges to decrease, such that the V-shaped deformable portions are forced to be folded inward toward the at least one bladder chamber, thereby reducing the space inside the at least one bladder chamber, squeezing the fluid in the at least one bladder chamber, and discharging the fluid from the at least one connector to the fluid supply system. When the main arm body is swung inward, the discharge of the fluid from the at least one bladder chambers of the knee booster is defined as the buffering action of the knee booster.

The at least one connector of the knee booster is connected to a fluid delivery tube of the fluid supply system carried by the user. By setting the fluid supply system, the fluid can freely enter and exit the at least one bladder chamber, enabling the user to wear the knee brace on the thigh and calf in either a standing or sitting position. When the user walks normally, the flow of the fluid in and out of the at least one bladder chambers in the bladder body of the knee booster can be regulated. This provides flexible protection and limitation for the movement of the knee joint. Similarly, during the process of a user’s squatting or sitting down, the discharge of the fluid from the at least one bladder chamber can be regulated through the buffering action generated by the knee booster, thus mitigating excessive stress on the knee joint’s myofascial tissue. The support capacity of the buffering effect can be adjusted by regulating the flow rate of fluid discharge to align with knee rehabilitation or training therapy. Further, when the user squats down or stands up from a squatting position, the fluid supply system pressurizes the at least one bladder chamber inside the bladder body of the knee booster, enabling the knee booster to generate the boosting action, which pulls up the user’s thigh via the main arm body of the thigh support, thereby assisting the knee joint in exerting force. The user can stand up without the aid of assistive devices. Furthermore, the magnitude of the thrust generated by the boosting action can be adjusted by varying the fluid pressure, thereby aligning with knee rehabilitation or training therapy. That is to say, the overall structure of the knee brace uses the elbow hinge assembly on the bladder body of the knee booster to serve as the energy generator and energy transmitter when the knee booster generates the buffering action or the boosting action. When the knee brace is used for rehabilitation or training therapy for patients with knee joint injuries, the knee booster generates buffering support energy or boosting energy, effectively achieving multiple benefits such as providing flexible protection and limitation for the movement of the knee joint when the user walks, flexibly reducing the force on the knee joint during squatting or sitting, and assisting the knee joint in exerting force during squatting, sitting, or standing.

Preferably, a partition is disposed inside the bladder body of the knee booster and located between the lateral sides for dividing the at least one bladder chamber into two bladder chambers. The actuating link and the limiting link each have the connector. The connector on the actuating link and the connector on the limiting link each have the passage therein. The passages of the connectors on the actuating link and the limiting link are in fluid communication with the corresponding bladder chambers, enabling the fluid in the bladder chambers to enter and exit through the connector on the actuating link and the connector on the limiting link. During the pressurization of the two bladder chambers, each elbow hinge of the elbow hinge assembly generates maximum thrust when the angle between the first hinge element and the second hinge element is close to or equal to 180 degrees. The boosting action of the knee booster is generated by selectively pressurizing the two bladder chambers in stages, accelerating the main arm body of the thigh support to respond to the thrust generated by the boosting action of the knee booster. The connecting portion of the thigh support has a through hole close to the first fixing seat. A fluid delivery tube is inserted through the through hole, which can provide directional guidance and positioning. When the connector on the actuating link and the connector on the limiting link are connected with the respective fluid delivery tubes, the two fluid delivery tubes are integrated and connected to the fluid supply system.

Preferably, the elbow hinges are pivotally connected to one another, and the first hinge element and the second hinge element of each of the elbow hinges are pivotally connected in a V-shape. Two ends of each of the first hinge element and the second hinge element have a male hinge portion and a female hinge portion that are mated with each other and pivotally connected by a pivot rod passing through the male hinge portion and the female hinge portion. The male hinge portion and the female hinge portion each have an engaging edge on mating end faces thereof for restricting the V-shaped angle between the first hinge element and the second hinge element of each of the elbow hinges to not exceed 180 degrees. The first hinge element and the second hinge element of each of the elbow hinges are restricted when the V-shaped angle between the first hinge element and the second hinge element is close to 180 degrees, preventing the elbow hinges from flipping outward and causing a thrust failure anomaly when the knee booster generates the boosting action.

Preferably, the bladder body of the knee booster includes a box-like bladder member and a cover. The cover and a closed side of the box-like bladder member serve as the lateral sides and seal the bladder body to form the at least one bladder chamber. When the box-like bladder member is to be formed using a mold, the hinge assembly is first embedded in the mold and then the box-like bladder member is formed by injection molding, such that the elbow hinge assembly is attached to the actuating end portion, the limiting end portion and the plurality of deformable portions. A plurality of perforations and a plurality of grooves are formed on the respective surfaces of the plurality of elbow hinges, the actuating link, and the limiting link of the elbow hinge assembly. In the molding process of the box-like bladder member, the material passes through the perforations to fill the grooves, forming a composite structure, such that the elbow hinge assembly will not detach from the bladder body easily. Furthermore, the cover and the closed side of the box-like bladder member serve as the lateral sides. A plurality of recessed fold portions are formed on the surfaces of the pair of lateral sides, respectively. The recessed fold portions are each recessed inward toward the at least one bladder chamber. By allowing inward deformation and outward expansion through the plurality of recessed fold portions, when the knee booster repeatedly performs the buffering action and the boosting action, the recessed fold portions can prevent the pair of lateral sides from tearing due to excessive tensile fatigue.

Preferably, the main arm body of the thigh support has a concave surface. The concave surface allows attachment to the user’s thigh, enabling the main arm body to be securely fastened to the user’s thigh using straps. The main arm body has a plurality of strap holes on two sides thereof, allowing straps to pass through the strap holes for securing the main arm body to the user’s thigh easily.

Preferably, the upper end of the limiting arm of the calf support is pivotally connected to the bottom end of the second fixing seat through a pivot shaft that is perpendicular to the pivot. When the user wears the knee brace and walks at a normal pace, the limiting arm is allowed to swing left and right at the bottom end of the second fixing seat. This enables the user’s calf to move without being subjected to excessive or unnecessary constraint or restriction from the limiting arm.

Preferably, the support portion formed at the lower end of the limiting arm of the calf support is an arc-shaped frame configured for resting against the dorsal side of the user’s calf. Either end of the support portion has a strap slot. The support portion can be tied to the user’s calf using a strap passing through the strap slot, thereby preventing the support portion from moving backward away from the user’s calf.

Alternatively, the elbow hinges of the elbow hinge assembly are pivotally connected to one another by a pivot rod, and the pivot point of the pivot rod is defined as an elbow pivot point. Two ends of the pivot rod at the elbow pivot point pass through the pair of lateral sides. A U-shaped fixing bracket is fixedly connected to the two ends of the pivot rod. The U-shaped fixing bracket includes a support rod and a pair of extension portions extending from two ends of the support rod and each having a fixing hole formed in a front end thereof and being secured to the two ends of the pivot rod, such that the pair of extension portions are attached to the surfaces of the lateral sides, and the support rod supports the inner periphery at a specific point, thereby ensuring that the distance between the elbow pivot point and the center point remains constant. When the at least one bladder chamber in the bladder body expands, the distance between the elbow pivot point and the center point remains unchanged. When the knee booster generates the boosting action, each elbow pivot point serves as the support point at either end of each elbow hinge, thereby enabling the V-shaped angle between the first hinge element and the second hinge element of each elbow hinge to expand smoothly and effectively forming a stable thrust concentrated towards the actuating link.

Preferably, when the V-shaped angle between the first hinge element and the second hinge element of each of the elbow hinges is close to 180 degrees, a fabric cover is wrapped around the surface of the knee booster. The fabric cover is made of a flexible and tear-resistant material, which can be used to restrict the shape of the bladder body when it expands. The fabric cover restricts the V-shaped angle between the first hinge element and the second hinge element from exceeding 180 degrees when the first hinge element and the second hinge element are unfolded. When the knee booster generates the boosting action, the thrust from each elbow hinge can be transmitted and concentrated to the actuating link stably and effectively. The elbow hinges of the elbow hinge assembly are pivotally connected to one another by a pivot rod, and the pivot point of the pivot rod is defined as an elbow pivot point. Two ends of the pivot rod at the elbow pivot point pass through the pair of lateral sides and the fabric cover. A U-shaped fixing bracket is fixedly connected to the two ends of the pivot rod. The U-shaped fixing bracket includes a support rod and a pair of extension portions extending from two ends of the support rod and each having a fixing hole formed in a front end thereof and being secured to the two ends of the pivot rod, such that the pair of extension portions are attached to the surface of the fabric cover, and the support rod supports the inner periphery at a specific point, thereby ensuring that the distance between the elbow pivot point and the center point remains constant. When the at least one bladder chamber in the bladder body expands, the distance between the elbow pivot point and the center point remains unchanged. When the knee booster generates the boosting action, each elbow pivot point serves as the support point at either end of each elbow hinge, in cooperation with the fabric cover to restrict the V-shaped angle between the first hinge element and the second hinge element from exceeding 180 degrees, thereby enabling the elbow hinge to generate a stable thrust concentrated towards the actuating link effectively.

### BRIEF DESCRIPTION OF THE DRAWINGS

FIG. 1 is a perspective view of the present invention;

FIG. 2 is an exploded view of the present invention;

FIG. 3 is a side view of the present invention;

FIG. 4 is a sectional view taken along line A-A in FIG. 3;

FIG. 5 is an enlarged view of circle B in FIG. 4;

FIG. 6 is a perspective view of the knee booster of the present invention;

FIG. 7 is an exploded view of the knee booster of the present invention;

FIG. 8 is a perspective view of the elbow hinge assembly of the present invention;

FIG. 9 is an exploded view of the elbow hinge of the present invention;

FIG. 10 is a schematic view of the present invention when in use;

FIG. 11 is a schematic view of the present invention when in use, illustrating that the fluid flows in and out of the bladder chamber of the knee booster;

FIG. 12 is a schematic view of the circuit when the fluid freely enters and exits the bladder chamber according to an embodiment of the hydraulic fluid supply system of the present invention;

FIG. 13 is a schematic view of the circuit when the fluid is discharged from the bladder chamber according to an embodiment of the hydraulic fluid supply system of the present invention;

FIG. 14 is a schematic view of the circuit when the fluid freely enters and exits the bladder chamber according to an embodiment of the gaseous fluid supply system of the present invention;

FIG. 15 is a schematic view of the circuit when the fluid is discharged from the bladder chamber according to an embodiment of the gaseous fluid supply system of the present invention;

FIG. 16 is a schematic view of the present invention, illustrating that the fluid in the bladder chamber of the booster device is discharged;

FIG. 17 is a schematic view of the present invention, illustrating that the fluid in the bladder chamber of the booster device is discharged when the user sits on the chair or squats;

FIG. 18 is a schematic view of the present invention when in use, illustrating that the user sits on the chair;

FIG. 19 is a schematic view of the circuit when one of the two bladder chambers is pressurized by the hydraulic fluid supply system of the present invention;

FIG. 20 is a schematic view of the circuit when the other of the two bladder chambers is pressurized by the hydraulic fluid supply system of the present invention;

FIG. 21 is a schematic view of the circuit when one of the two bladder chambers is pressurized by the gaseous fluid supply system of the present invention;

FIG. 22 is a schematic view of the circuit when the other of the two bladder chambers is pressurized by the gaseous fluid supply system of the present invention;

FIG. 23 is a schematic view of the present invention when one of the two bladder chambers is pressurized;

FIG. 24 is a schematic view of the present invention when the other of the two bladder chambers is pressurized;

FIG. 25 is a schematic view of the present invention when in use, illustrating that the user stands up with the aid of the knee booster;

FIG. 26 is a perspective view of the knee booster equipped with the U-shaped fixing bracket of the present invention;

FIG. 27 is a perspective view of the U-shaped fixing bracket of the present invention;

FIG. 28 is a sectional view of the present invention when the knee booster is equipped with the U-shaped fixing bracket;

FIG. 29 is a schematic view of the present invention, illustrating that the U-shaped fixing bracket of the knee booster restricts the elbow pivot point;

FIG. 30 is a perspective view of the knee booster covered with the fabric cover of the present invention;

FIG. 31 is a sectional view of the knee booster covered with the fabric cover of the present invention;

FIG. 32 is a sectional view of the present invention when the knee booster is covered with the fabric cover;

FIG. 33 is a schematic view of the present invention, illustrating that the unfolding angle between the first and second hinge elements is restricted when the knee booster is covered with the fabric cover;

FIG. 34 is a perspective view of the knee booster covered with the fabric cover and equipped with the U-shaped fixing bracket of the present invention;

FIG. 35 is a sectional view of the knee booster covered with the fabric cover and equipped with the U-shaped fixing bracket of the present invention;

FIG. 36 is a sectional view of the present invention when the knee booster is covered with the fabric cover and equipped with the U-shaped fixing bracket; and

FIG. 37 is a schematic view of the present invention, illustrating that the unfolding angle between the first and second hinge elements and the elbow pivot point are restricted when the knee booster is covered with the fabric cover and equipped with the U-shaped fixing bracket.

### DETAILED DESCRIPTION OF THE PREFERRED EMBODIMENTS

Embodiments of the present invention will now be described, by way of example only, with reference to the accompanying drawings.

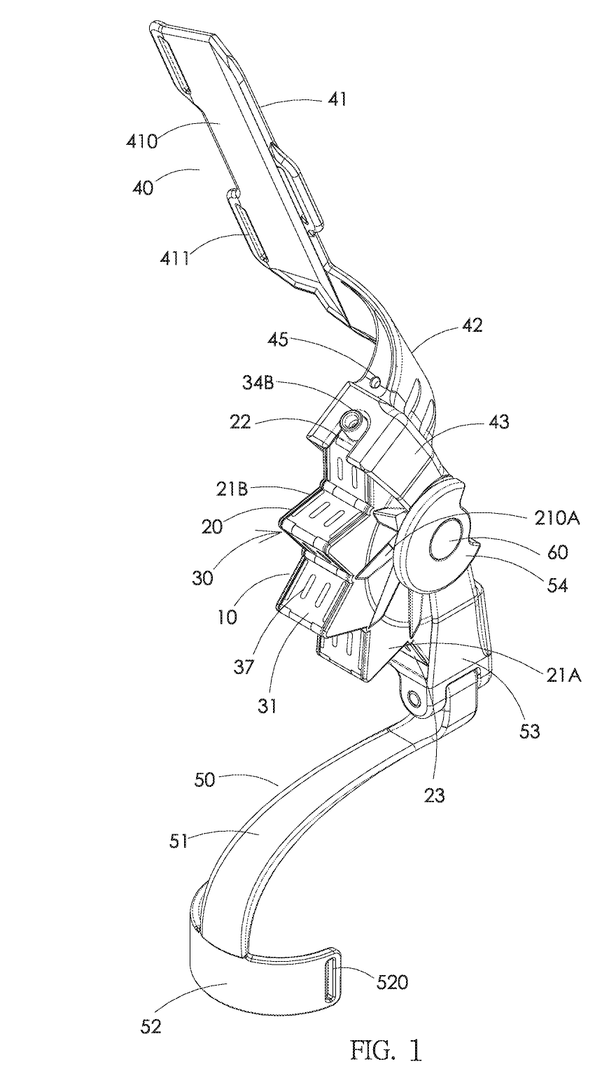

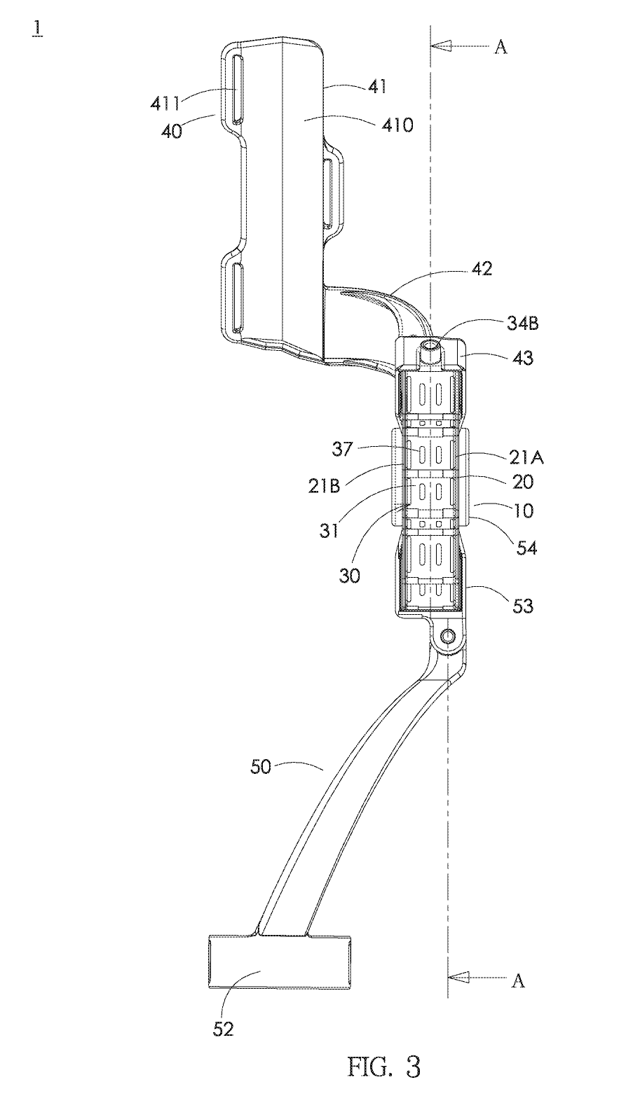

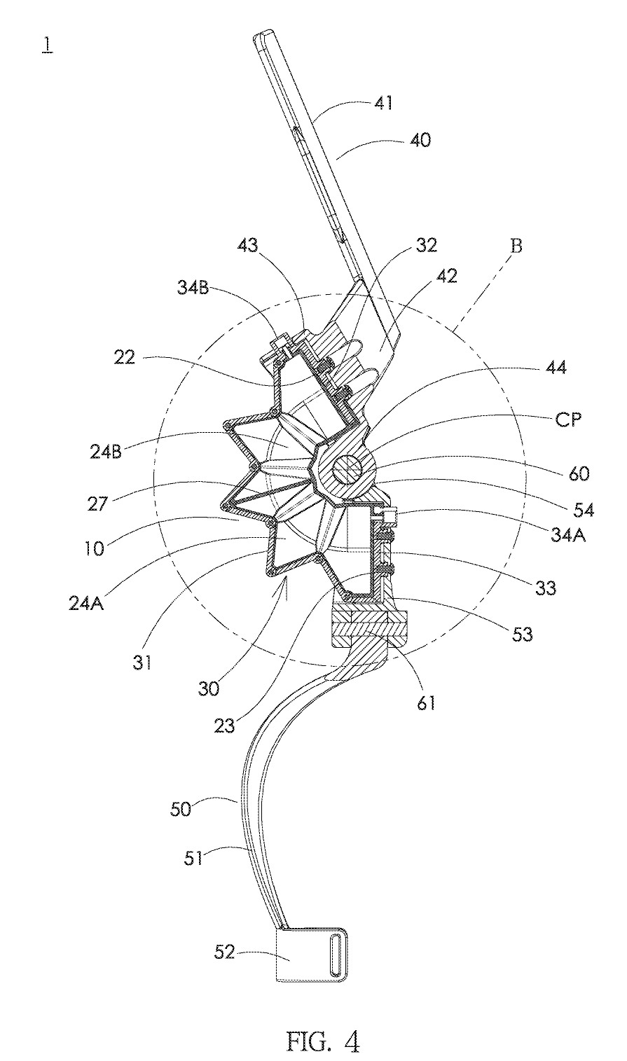

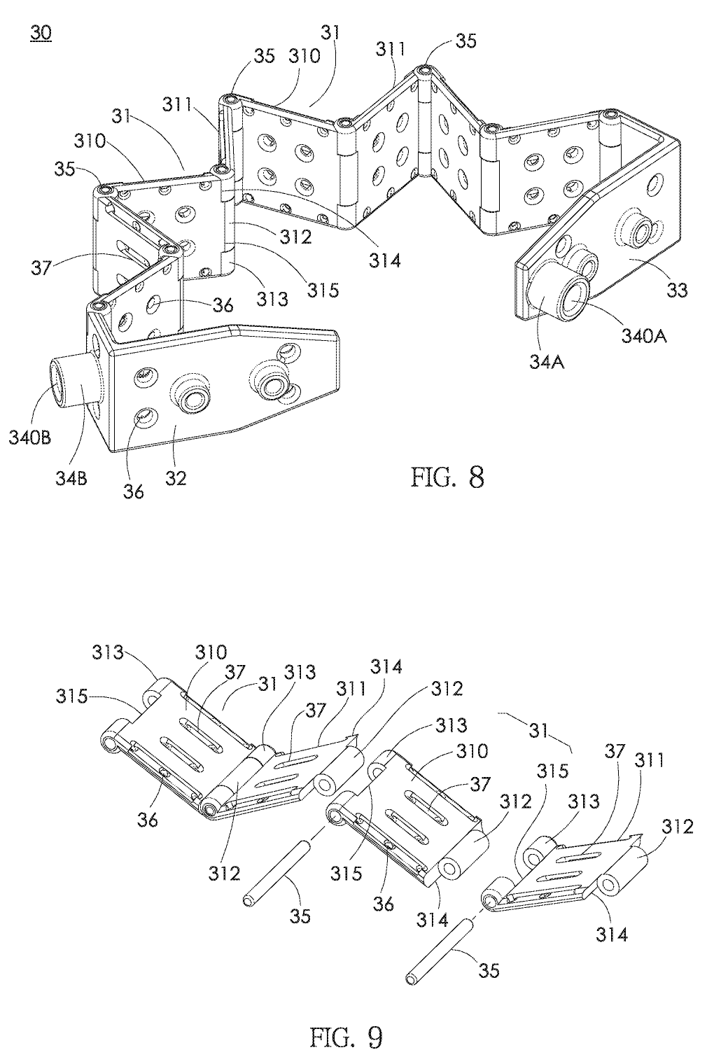

As shown in FIGS. 1-9, the present invention discloses a hinged knee brace 1, comprising a knee booster 10, a thigh support 40, and a calf support 50.

The knee booster 10 includes a fan-shaped bladder body 20. The bladder body 20 has a pair of lateral sides 21A and 21B, an actuating end portion 22 and a limiting end portion 23 at two ends of the lateral sides 21A and 21B, and at least one bladder chamber 24A, 24B inside the bladder body 20. The bladder body 20 is formed of an airtight material that may be rubber or silicone, or a material composed of highly resilient natural fibers or chemical fibers laminated with soft rubber or a film). The bladder body 20 is in the form of an expandable, deformable flexible bladder. The peripheries of the lateral sides 21A and 21B, between the actuating end portion 22 and the limiting end portion 23, are defined as an outer periphery 25 and an inner periphery 26 that share a common center point CP. The outer periphery 25 is formed with a continuous V-shaped corrugated configuration, thereby forming a plurality of deformable portions 250 that are approximately V-shaped. The inner periphery 26 is formed with approximately V-shaped folds, thereby forming a folded surface 260 that allows for unfolding and folding. An elbow hinge assembly 30 is disposed on (attached to) the plurality of deformable portions 250 of the outer periphery 25, the actuating end portion 22, and the limiting end portion 23. The elbow hinge assembly 30 includes a plurality of elbow hinges 31 that are pivotally connected to one another and correspond in number to the plurality of deformable portions 250. An actuating link 32 and a limiting link 33 are pivotally connected to two ends of the connected elbow hinges 31. Each of the plurality of elbow hinges 31 includes a first hinge element 310 and a second hinge element 311 that are pivotally connected in a V shape and have supporting rigidity (such as being made of metal or hard plastic), and is fitted onto (preferably, adhesively attached to) one of the deformable portions 250. The actuating link 32 is pivotally connected to one end of the connected elbow hinges 31, and is fitted onto (preferably, adhesively attached to) the actuating end portion 22 and has supporting rigidity (such as being made of metal or hard plastic). The limiting link 33 is pivotally connected to the other end of the connected elbow hinges 31, and is fitted onto (preferably, adhesively attached to) the limiting end portion 23 and has supporting rigidity (such as being made of metal or hard plastic). At least one connector 34A, 34B is disposed on at least one of the actuating link 32 on the actuating end portion 22 and the limiting link 33 on the limiting end portion 23 of the bladder body 20. The at least one connector 34A, 34B has a passage 340A, 340B therein. The passage 340A, 340B is in fluid communication with the at least one bladder chamber 24A, 24B, enabling a fluid in the at least one bladder chamber 24A, 24B to enter and exit through the at least one connector 34A, 34B.

The thigh support 40 is an arm-shaped frame having supporting rigidity (such as being made of metal or non-metal rigid plastic materials). The thigh support 40 includes a main arm body 41, a curved connecting portion 42, and a first fixing seat 43. The main arm body 41 is configured for being secured to the user’s thigh with a fastening strap. The connecting portion 42 extends from one side of the lower end of the main arm body 41 and is designed to match the curvature of the user’s thigh. The first fixing seat 43 (preferably, a frame-shaped seat) is formed on the front end of the connecting portion 42. The first fixing seat 43 is coupled with the actuating end portion 22 of the bladder body 20 and is fixedly connected (locked) to the actuating link 32. A first pivot seat 44, centered on the center point CP, is formed on the lower side of one end of the first fixing seat 43.

The calf support 50 is in the form of an arm body with tensile strength (such as being made of metal or non-metal rigid plastic materials). The calf support 50 includes a limiting arm 51, a support portion 52, and a second fixing seat 53. The support portion 52 extends from the lower end of the limiting arm 51 and is configured for holding the user’s calf in cooperation with a fastening strap. The second fixing seat 53 (preferably, a frame-shaped seat) is disposed on the upper end of the limiting arm 51. The second fixing seat 53 is coupled with the limiting end portion 23 of the bladder body 20 and is fixedly connected (locked) to the limiting link 33. A second pivot seat 54 is formed on one side of the second fixing seat 53, corresponding to the first pivot seat 44. The second pivot seat 54 is pivotally connected to the first pivot seat 44 through a pivot 60.

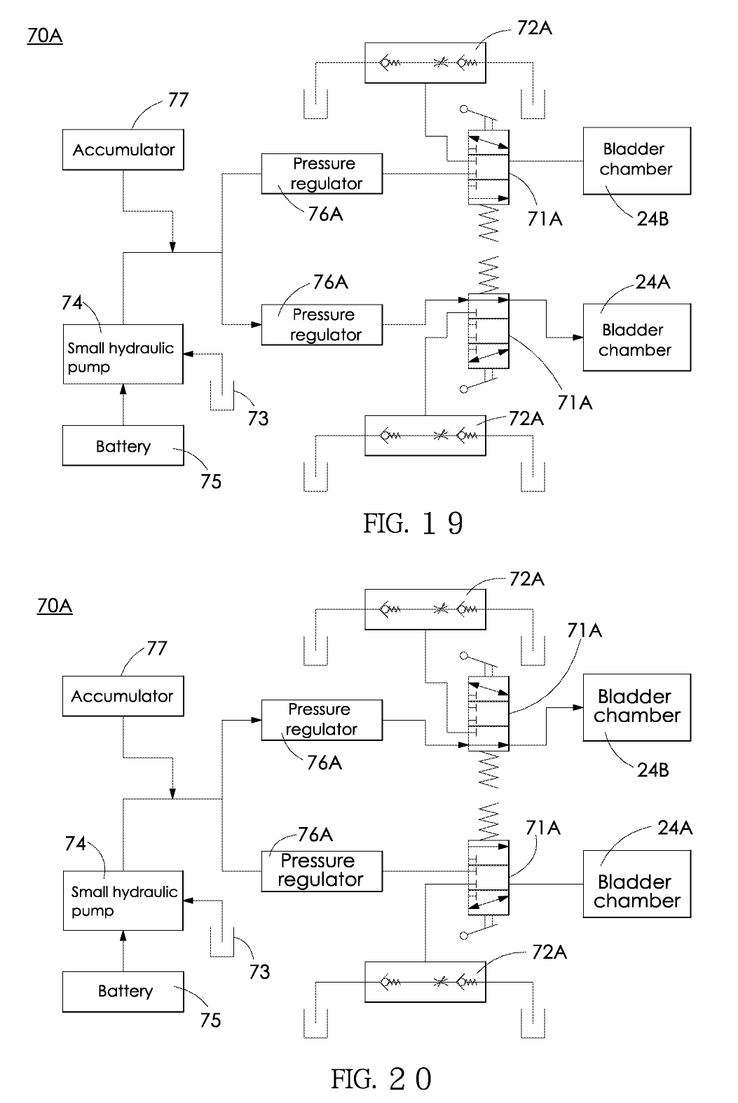

As shown in FIGS. 19, 20, 23, and 24, when a fluid (which may be a liquid or gas) is supplied from a fluid supply system 70A to the at least one bladder chamber 24A, 24B of the knee booster 10 via at least one connector 34A, 34B, the space inside the at least one bladder chamber 24A, 24B expands, enabling the plurality of deformable portions 250 of the bladder body 20 to expand and deform. Thus, the angle between the first hinge element 310 and the second hinge element 311 of each of the plurality of elbow hinges 31 increases, generating an accumulated thrust (a thrust supported by the stationary limiting link 33) that is concentrated on the actuating link 32, such that the main arm body 41 of the thigh support 40, pivoted about the pivot 60 (i.e., the center point CP), bears the thrust from the actuating link 32 and performs an outward swing motion (in the clockwise direction shown in the figures). The action of transmitting the thrust generated by the knee booster 10 to the main arm body 41 is defined as a boosting action of the knee booster 10. Conversely, as shown in FIGS. 13, 16, and 17, when an external force (such as the force exerted by the thigh when the user squats) is applied to the main arm body 41, causing the main arm body 41 to perform an inward swing motion (in the counterclockwise direction shown in the figures), the actuating link 32 is driven to push the plurality of elbow hinges 31 so that the angle between the first hinge element 310 and the second hinge element 311 of each of the plurality of elbow hinges 31 to decrease, such that the V-shaped deformable portions 250 are forced to be folded inward toward the at least one bladder chamber 24A, 24B, thereby reducing (decreasing) the space inside the at least one bladder chamber 24A, 24B, squeezing the fluid in the at least one bladder chamber 24A, 24B, and discharging the fluid from the at least one connector 34A, 34B to the fluid supply system 70A. When the main arm body 41 is swung inward, the discharge of the fluid from the at least one bladder chambers 24A, 24B of the knee booster 10 is defined as the buffering action of the knee booster 10.

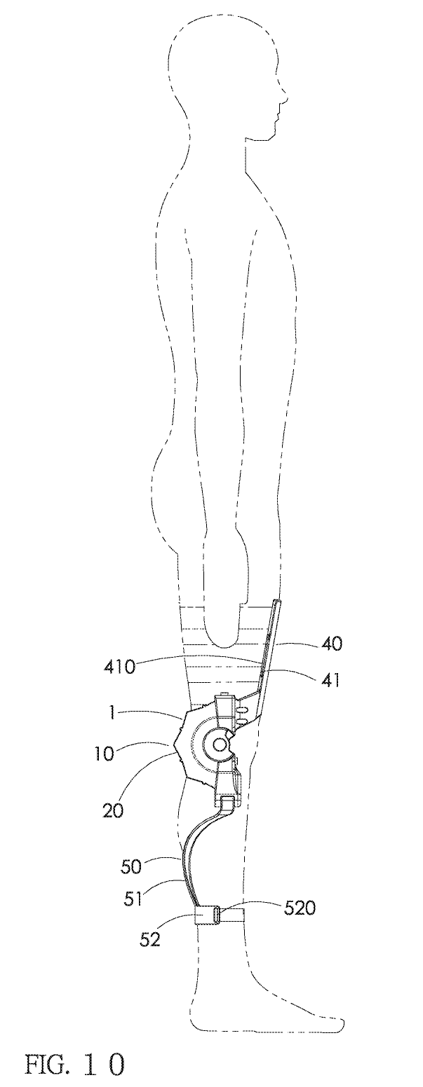

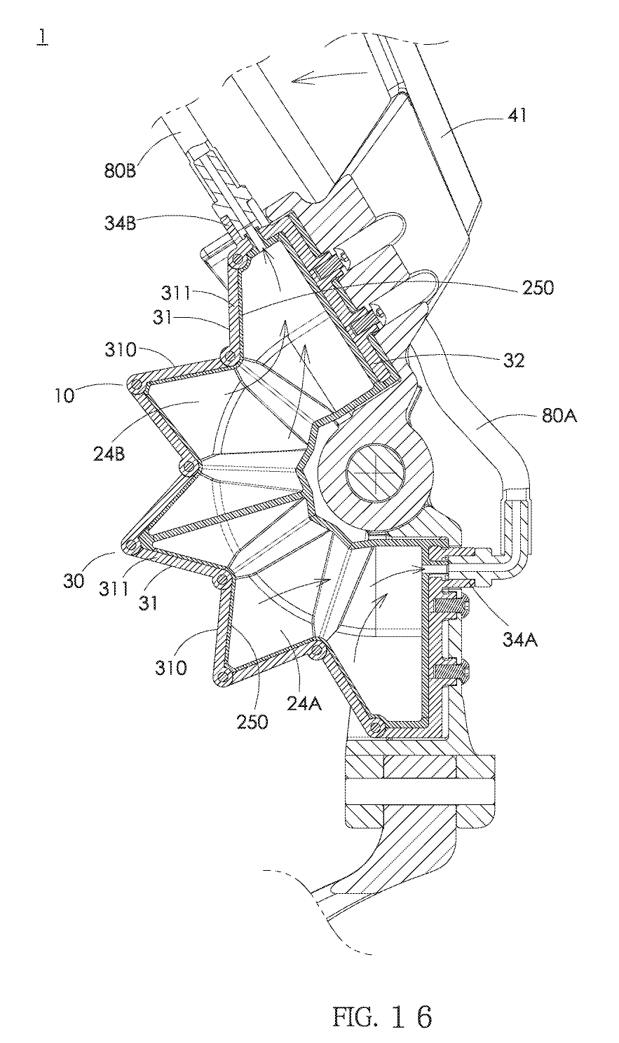

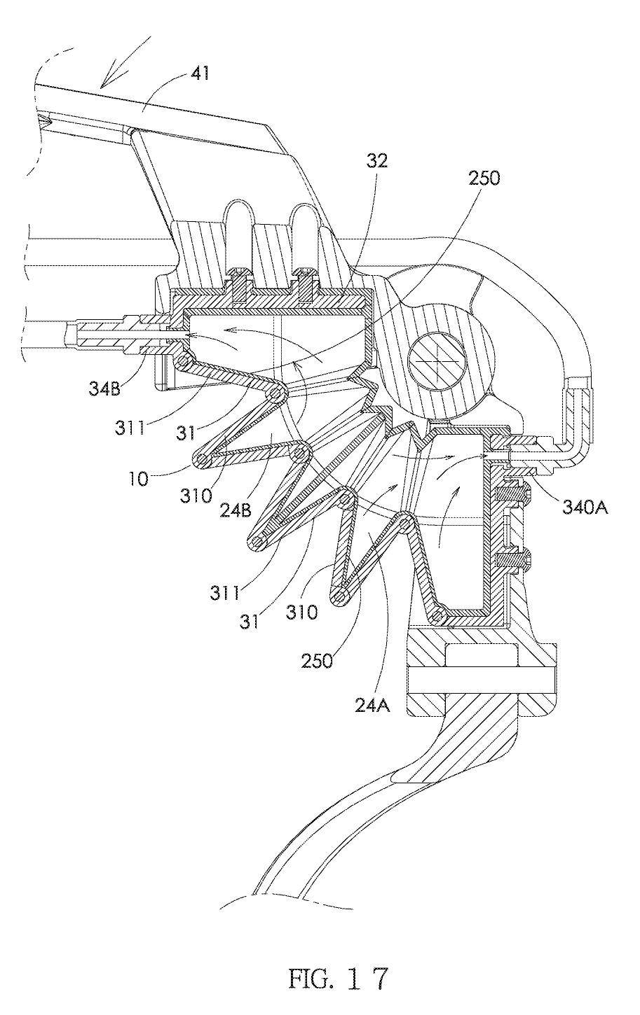

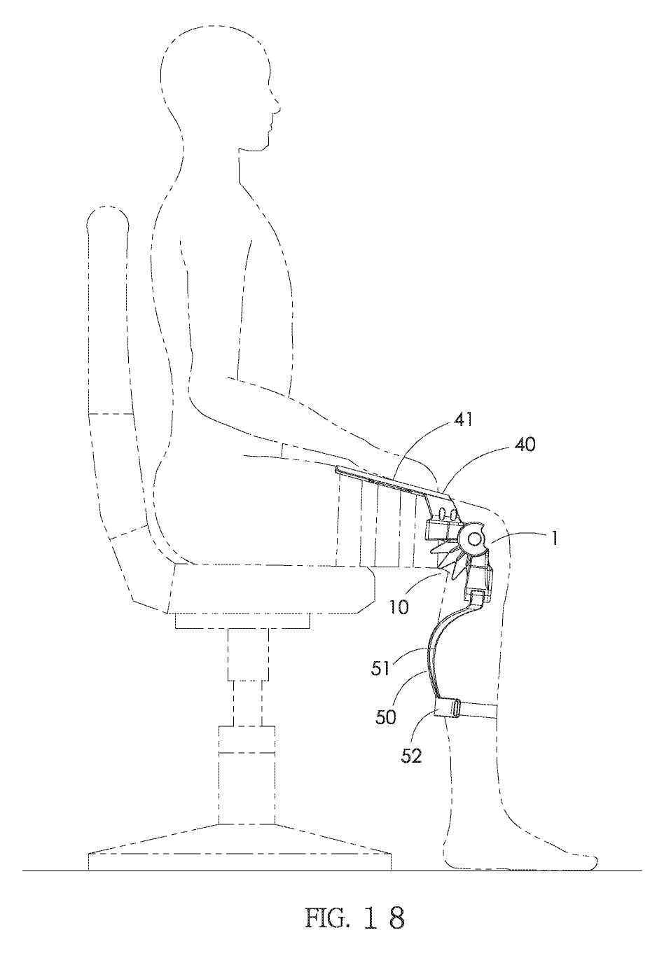

As shown in FIGS. 10, 11, and 12, the at least one connector 23A, 23B of the knee booster 10 is connected to a fluid delivery tube 80A, 80B of the fluid supply system 70A (not shown in FIG. 10) carried by the user. By setting (switching) the fluid supply system 70A, the fluid can freely enter and exit the at least one bladder chamber 24A, 24B, enabling the user to wear the knee brace 1 on the thigh and calf in either a standing or sitting position. In this state, when the user walks normally, the flow of the fluid in and out of the at least one bladder chambers 24A, 24B in the bladder body 20 of the knee booster 10 can be regulated. This provides flexible protection and limitation for the movement of the knee joint. Similarly, in this state, as shown in FIGS. 13, 16, 17 and 18, during the process of a user’s squatting or sitting down and upon reaching the final position, the discharge of the fluid from the at least one bladder chamber 24A, 24B can be regulated through the buffering action generated by the knee booster 10, thus mitigating excessive stress on the knee joint’s myofascial tissue. The support capacity of the buffering effect can be adjusted by regulating the flow rate of fluid discharge to accommodate knee rehabilitation or training therapy. Further, as shown in FIGS. 19, 20, 23, 24 and 25, when the user squats down or stands up from a squatting position, the fluid supply system 70A pressurizes the at least one bladder chamber 24A, 24B inside the bladder body 20 of the knee booster 10, enabling the knee booster 10 to generate the boosting action, which pulls up the user’s thigh via the main arm body 41 of the thigh support 40, thereby assisting the knee joint in exerting force. The user can stand up without the aid of assistive devices. Furthermore, the magnitude of the thrust generated by the boosting action can be adjusted by varying the fluid pressure, thereby aligning with knee rehabilitation or training therapy. That is to say, as shown in FIGS. 1, 2, 5, 16 and 17, the overall structure of the knee brace 1 uses the elbow hinge assembly 30 on the bladder body 20 of the knee booster 10 to serve as the energy generator and energy transmitter when the knee booster 10 generates the buffering action or the boosting action. As shown in FIG. 10, when the knee brace 1 is used for rehabilitation or training therapy for patients with knee joint injuries, as shown in FIGS. 16, 23 and 24, the knee booster 10 generates buffering support energy or boosting energy, effectively achieving multiple benefits such as providing flexible protection and limitation for the movement of the knee joint when the user walks, flexibly reducing the force on the knee joint during squatting or sitting, and assisting the knee joint in exerting force (boosting) during squatting, sitting, or standing.

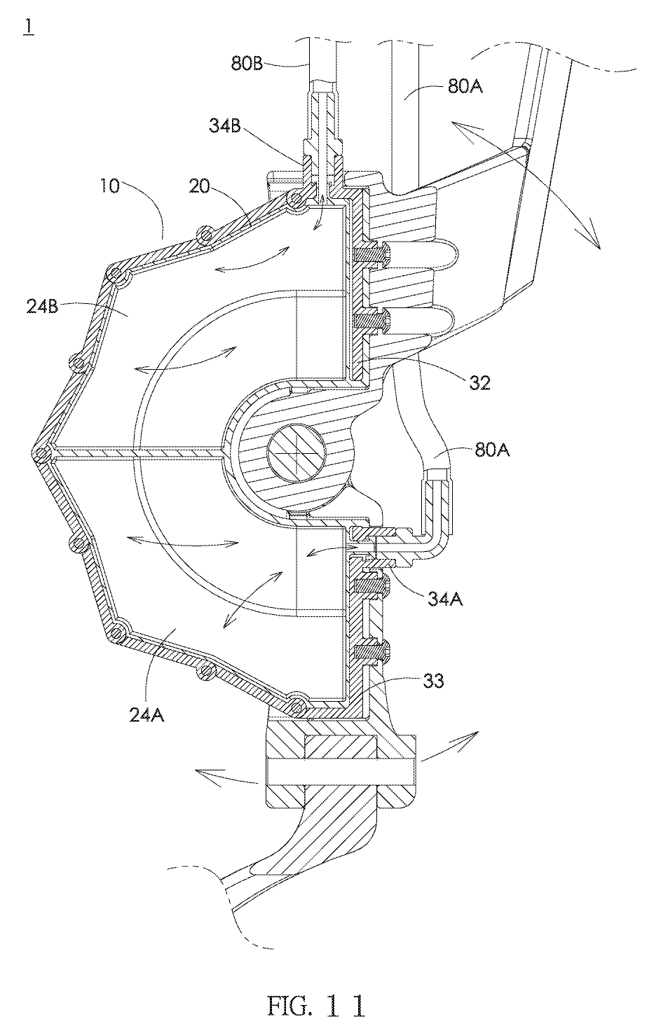

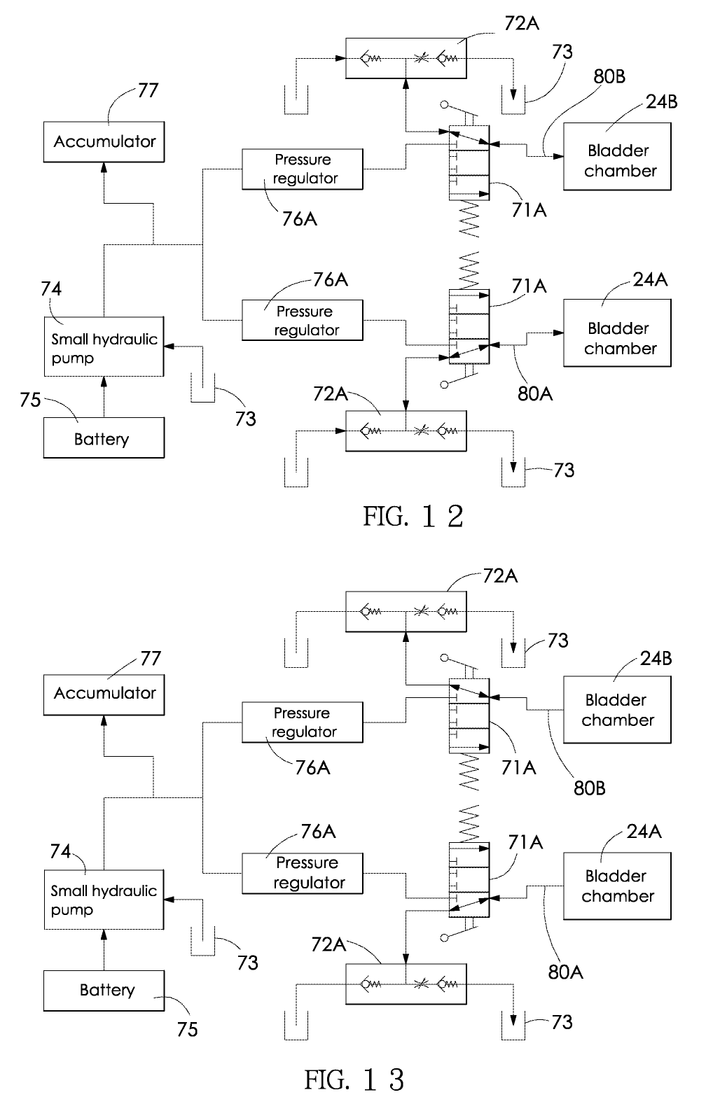

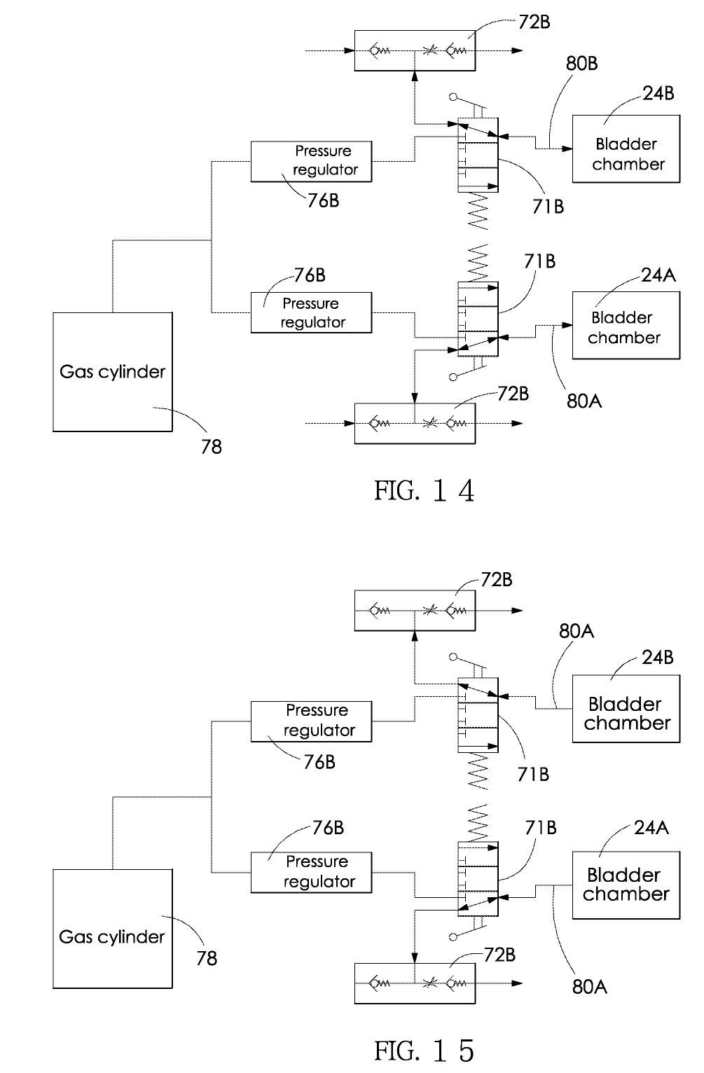

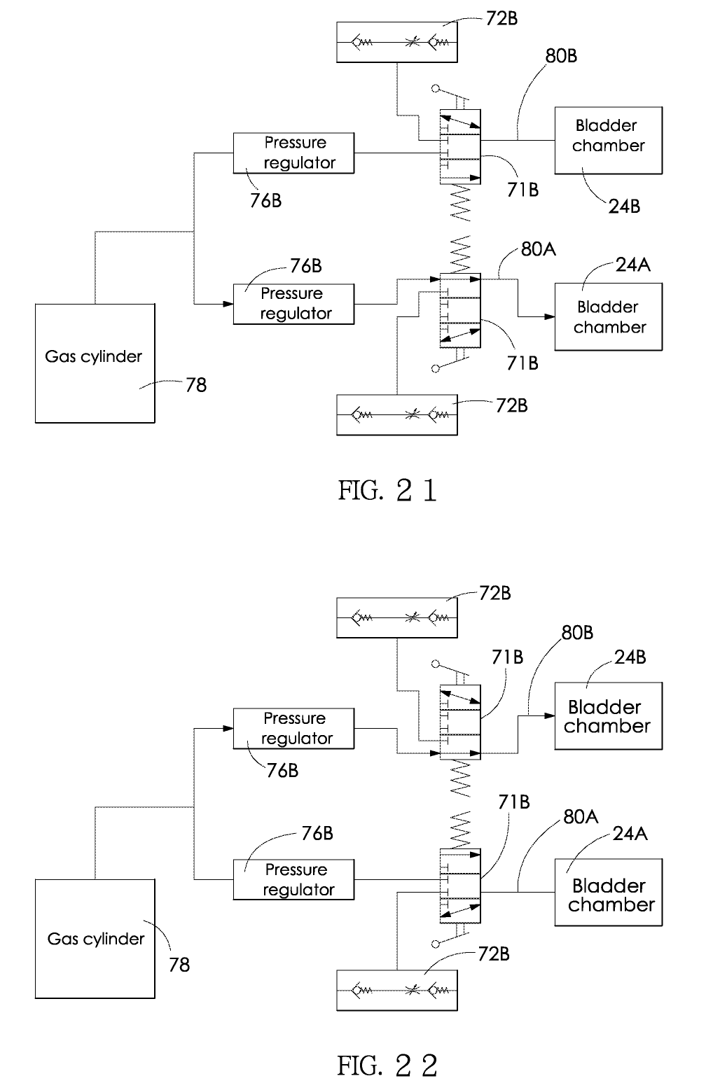

As shown in FIGS. 11 and 12, for the implementation of the fluid supply system 70A, it is feasible to integrate it into a backpack or belt (not shown) and wear it on the user’s body (not shown). The fluid supply system 70A, which can be selected to supply a hydraulic fluid, is controlled by a manually or electrically controlled switch valve 71A to switch (set) the liquid in the at least one bladder chamber 24A, 24B of the knee booster 10 to be in communication with a reservoir or oil tank 73 via a flow controller 72A, allowing free entry and exit of the liquid. As shown in FIGS. 13, 16 and 17, by regulating (or restricting) the inflow and outflow of the liquid through the flow controller 72A, the knee booster 10 can provide either a supportive or restrictive buffering action for the movement of the knee joint. When there is a need for the knee booster 10 to generate the boosting action, as shown in FIGS. 19, 20, 23 and 24, the switching action of the switch valve 71A directs hydraulic fluid delivered by a small hydraulic pump 74 (such as a small plunger-type hydraulic pump powered by a battery 75) through a pressure regulator 76A. The regulated fluid then hydraulically pressurizes the at least one bladder chamber 24A, 24B. The hydraulic fluid discharged from the small hydraulic pump 74 can be directed into an accumulator 77 for storage during periods when there is no need for the knee booster 10 to generate the boosting action (idle periods). When needed, the hydraulic fluid is then released from the accumulator 77 to meet the timing requirements for the knee booster 10 to generate the boosting action. Furthermore, as shown in FIGS. 11 and 14, the fluid supply system 70B, which can be selected to supply a gaseous fluid (e.g., for the treatment of patients with non-severe tears of the knee joint), is controlled by a manually controlled switch valve 71B to switch the gaseous fluid in the at least one bladder chamber 24A, 24B of the knee booster 10 to be in communication with the outside via a flow controller 72B, allowing free entry and exit of the gas. As shown in FIGS. 15, 16 and 17, by regulating (or restricting) the inflow and outflow of the gas through the flow controller 72B, the knee booster 10 can provide either a supportive or restrictive buffering action for the movement of the knee joint. When there is a need for the knee booster 10 to generate the boosting action, as shown in FIGS. 21, 22, 23 and 24, the switching action of the switch valve 71B directs the gaseous fluid released from a gas cylinder 78 (such as a carbon dioxide cylinder used in paintball guns) through a pressure regulator 76B to inflate the at least one bladder chamber 24A, 24B, enabling the knee booster 10 to generate the boosting action.

As shown in FIG. 10, the knee brace 1 described above can be used on the thigh and calf. It can be directly attached to the exposed thigh and calf or the surface of the trouser leg. When in use, the knee brace 1 is secured to the thigh and calf positions. Additionally, the knee brace 1 may be used in cooperation with a knee elastic sleeve (band), liner, or pad (not shown in the figure) to enhance the comfort of the main arm body 41 of the thigh support 40 when secured to the thigh with straps.

Furthermore, as shown in FIGS. 1, 2, 4 and 10, the knee brace 1 implemented in the figures is an example of the knee brace 1 for use on the right leg. With the same technical solution, by modifying the connecting portion 42 extending from the lower end of the other side of the main arm body 41 of the thigh support 40 and the first fixing seat 43 and by reversing the direction of the limiting arm 51 of the calf support 50, another embodiment of the knee brace 1 is used for the left leg. Depending on the treatment for an injury to the left or right knee joint, the user may select the knee brace 1 designed for use on either the left leg or the right leg. If both knee joints are injured, the user may wear the corresponding knee braces 1 for the left and right legs simultaneously.

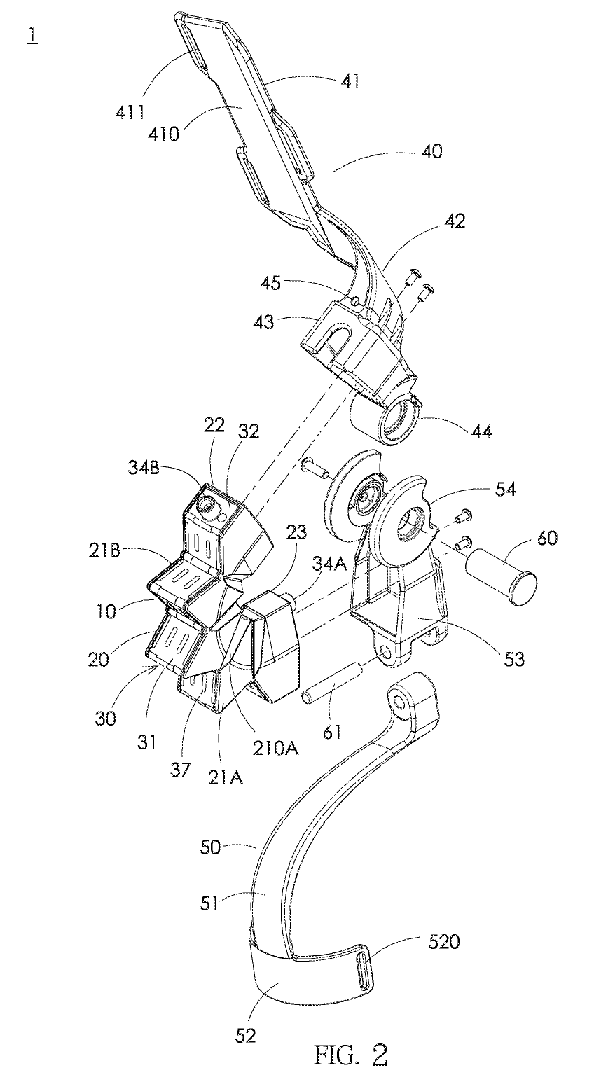

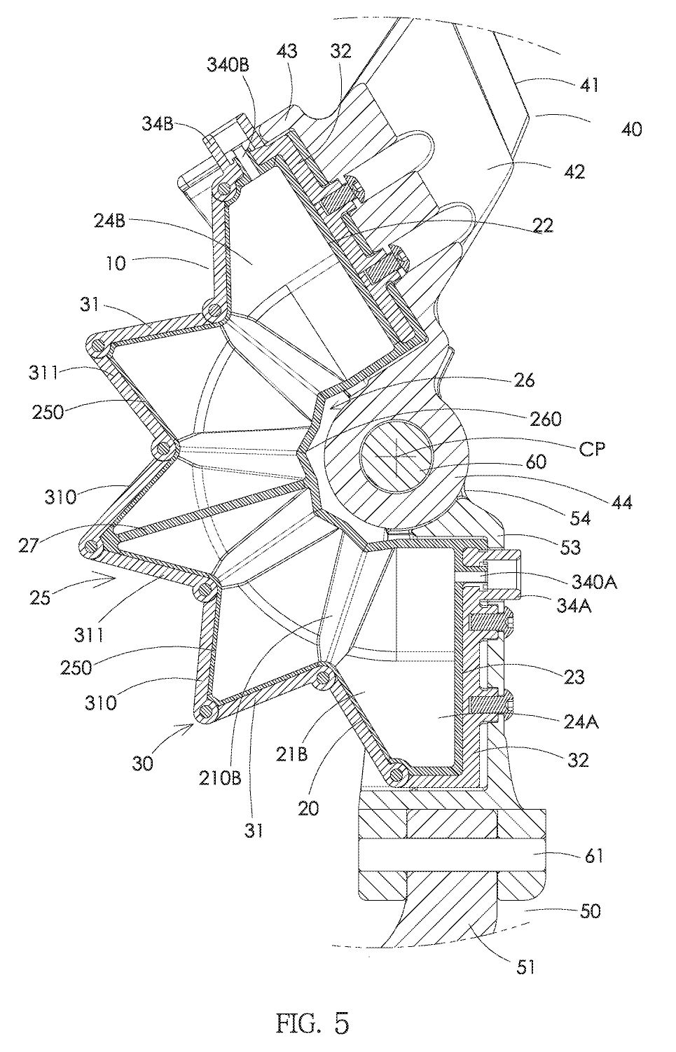

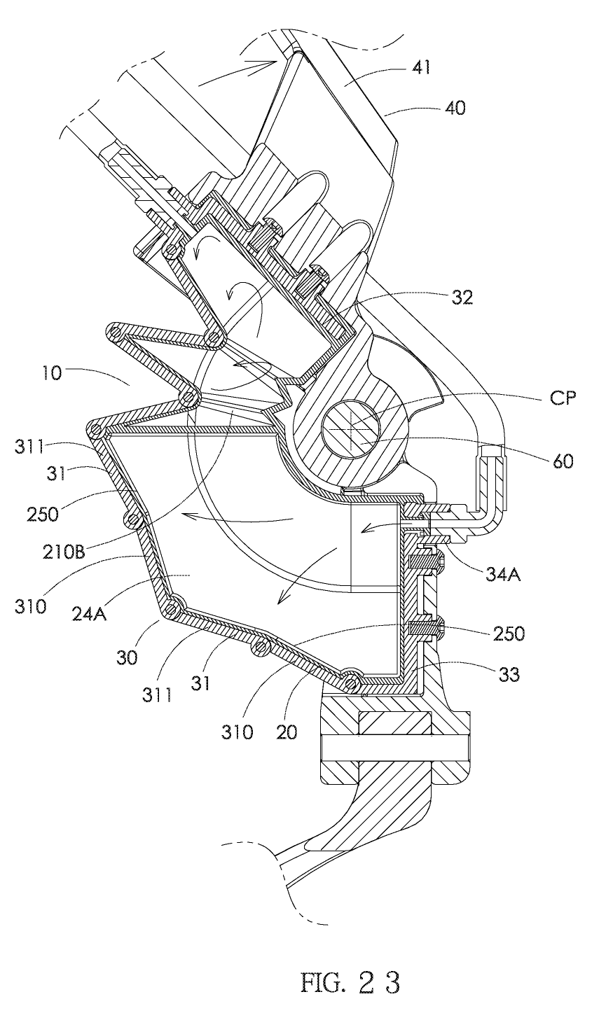

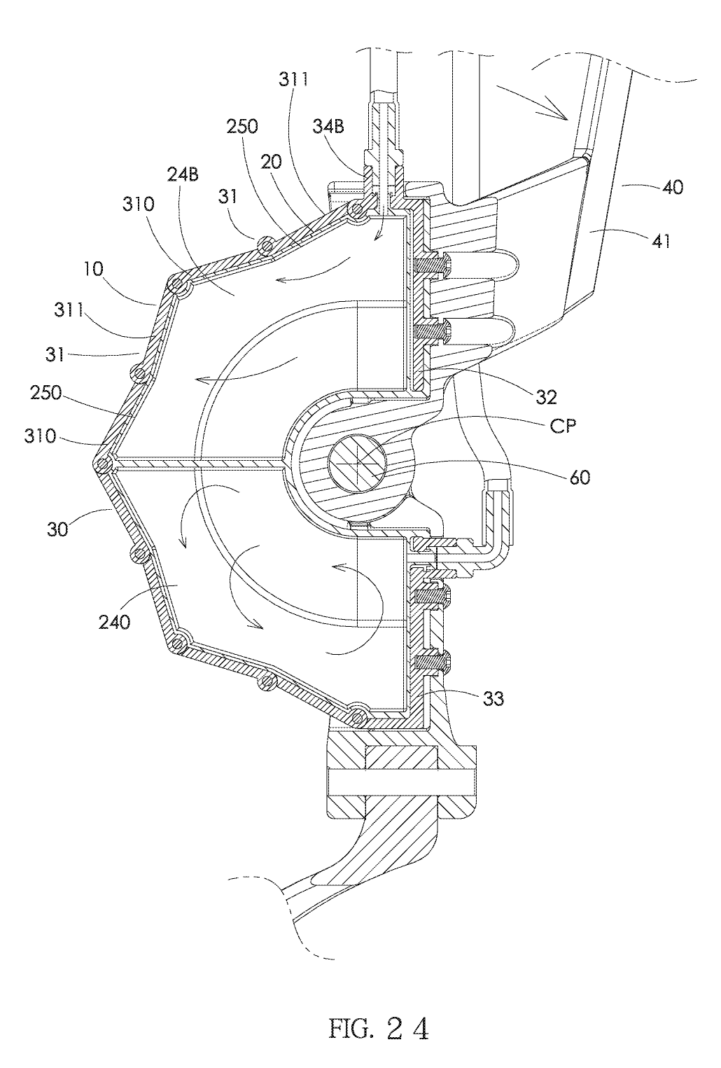

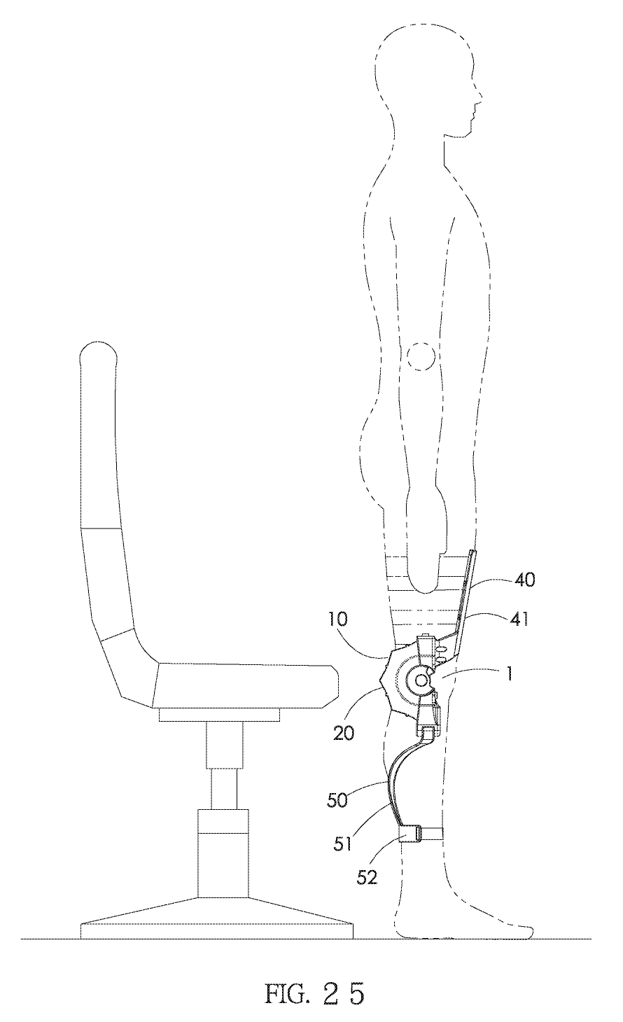

According to the above embodiment, preferably, as shown in FIG. 5, a partition 27 is disposed inside the bladder body 20 of the knee booster 10 and located between the lateral sides 21A and 21B for dividing the at least one bladder chamber 24A, 24B into two bladder chambers 24A and 24B. The actuating link 32 and the limiting link 33 each have the connector 34A, 34B. The connector 34B on the actuating link 32 and the connector 34A on the limiting link 33 each have the passage 340A, 340B therein. The passages 340A and 340B of the connectors 34A and 34B on the actuating link 32 and the limiting link 33 are in fluid communication with the corresponding bladder chambers 24A, 24B, enabling the fluid in the bladder chambers 24A and 24B to enter and exit through the connector 34B on the actuating link 32 and the connector 34A on the limiting link 33. As shown in FIGS. 23 and 24, during the pressurization of the two bladder chambers 24A and 24B, each elbow hinge 31 of the elbow hinge assembly 30 generates maximum thrust when the angle between the first hinge element 310 and the second hinge element 311 is close to or equal to 180 degrees. The boosting action of the knee booster 10 is generated by selectively pressurizing the two bladder chambers 24A and 24B in stages, accelerating the main arm body 41 of the thigh support 40 to respond to the thrust generated by the boosting action of the knee booster 10. As shown in FIGS. 1 and 2, the connecting portion 42 of the thigh support 40 has a through hole 45 close to the first fixing seat 43. As shown in FIGS. 1 and 11, the fluid delivery tube 80A is inserted through the through hole 45, which can provide directional guidance and positioning. When the connector 34B on the actuating link 32 and the connector 34A on the limiting link 33 are connected with the respective fluid delivery tubes 80A and 80B, the two fluid delivery tubes 80A and 80B are integrated and connected to the fluid supply system.

According to the above embodiment, preferably, as shown in FIGS. 5, 8 and 9, the elbow hinges 31 are pivotally connected to one another, and the first hinge element 310 and the second hinge element 311 of each of the elbow hinges 31 are pivotally connected in a V-shape. Two ends of each of the first hinge element 310 and the second hinge element 311 have a male hinge portion 312 and a female hinge portion 313 that are mated with each other and pivotally connected by a pivot rod 35 passing through the male hinge portion 312 and the female hinge portion 313. The male hinge portion 312 and the female hinge portion 313 each have an engaging edge 314, 315 on mating end faces thereof for restricting the V-shaped angle between the first hinge element 310 and the second hinge element 311 of each of the elbow hinges 31 to not exceed 180 degrees. As shown in FIGS. 23 and 24, the first hinge element 310 and the second hinge element 311 of each of the elbow hinges 31 are restricted when the V-shaped angle between the first hinge element 310 and the second hinge element 311 is close to 180 degrees, preventing the elbow hinges 31 from flipping outward and causing a thrust failure anomaly when the knee booster 10 generates the boosting action.

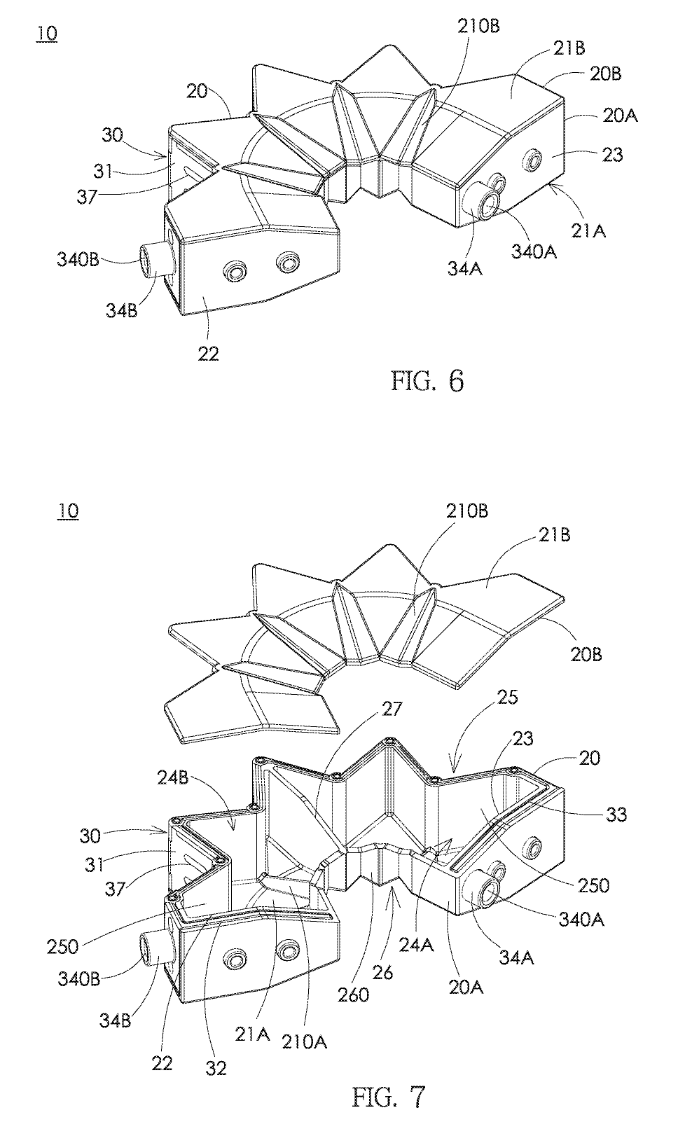

According to the above embodiment, preferably, as shown in FIGS. 5, 6 and 7, the bladder body 20 of the knee booster 10 includes a box-like bladder member 20A and a cover 20B covering (being attached to) an open side of the box-like bladder member 20A. The box-like bladder member 20A has the actuating end portion 22, the limiting end portion 23, and the plurality of deformable portion 250. The cover 20B and a closed side of the box-like bladder member 20A serve as the lateral sides 21A and 21B and seal the bladder body 20 to form the at least one bladder chamber 24A, 24B. When the box-like bladder member 20A is to be formed using a mold (not shown, such as an injection molding mold), the hinge assembly 30 is first embedded in the mold and then the box-like bladder member 20A is formed by injection molding, such that the elbow hinge assembly 30 is attached to (coupled to) the actuating end portion 22, the limiting end portion 23 and the plurality of deformable portions 250. As shown in FIG. 8, a plurality of perforations 36 and a plurality of grooves 37 are formed on the respective surfaces of the plurality of elbow hinges 31, the actuating link 32, and the limiting link 33 of the elbow hinge assembly 30. As shown in FIGS. 7 and 8, in the molding process of the box-like bladder member 20A, the material passes through the perforations 36 to fill the grooves 37, forming a composite structure, such that the elbow hinge assembly 30 will not detach from the bladder body 20 easily. Furthermore, as shown in FIGS. 6 and 7, the cover 20B and the closed side of the box-like bladder member 20A serve as the lateral sides 21A and 21B. A plurality of recessed fold portions 210A and 210B are formed on the surfaces of the pair of lateral sides 21A and 21B, respectively. The recessed fold portions 210A and 210B are each recessed inward toward the at least one bladder chamber 24A, 24B. By allowing inward deformation and outward expansion through the plurality of recessed fold portions 210A and 210B, when the knee booster 10 repeatedly performs the buffering action and the boosting action, the recessed fold portions 210A and 210B can prevent the pair of lateral sides 21A and 21B from tearing due to excessive tensile fatigue.

According to the above embodiment, preferably, as shown in FIGS. 1 and 2, the main arm body 41 of the thigh support 40 has a concave surface 410. As shown in FIGS. 1 and 10, the concave surface 410 allows attachment to the user’s thigh, enabling the main arm body 41 to be securely fastened to the user’s thigh using straps. As shown in FIGS. 1 and 2, the main arm body 41 has a plurality of strap holes 411 on two sides thereof, allowing straps to pass through the strap holes 411 for securing the main arm body 41 to the user’s thigh easily.

According to the above embodiment, preferably, as shown in FIGS. 1 and 2, the upper end of the limiting arm 51 of the calf support 50 is pivotally connected to the bottom end of the second fixing seat 53 through a pivot shaft 61 that is perpendicular to the pivot 60. As shown in FIG. 10, when the user wears the knee brace 1 and walks at a normal pace, the limiting arm 51 is allowed to swing left and right at the bottom end of the second fixing seat 53. This enables the user’s calf to move without being subjected to excessive or unnecessary constraint or restriction from the limiting arm 51.

According to the above embodiment, preferably, as shown in FIGS. 1 and 2, the support portion 52 formed at the lower end of the limiting arm 51 of the calf support 50 is an arc-shaped frame configured for resting against the dorsal side of the user’s calf. Either end of the support portion 52 has a strap slot 520. As shown in FIG. 10, the support portion 52 can be tied to the user’s calf using a strap passing through the strap slot 520, thereby preventing the support portion 52 from moving backward away from the user’s calf.

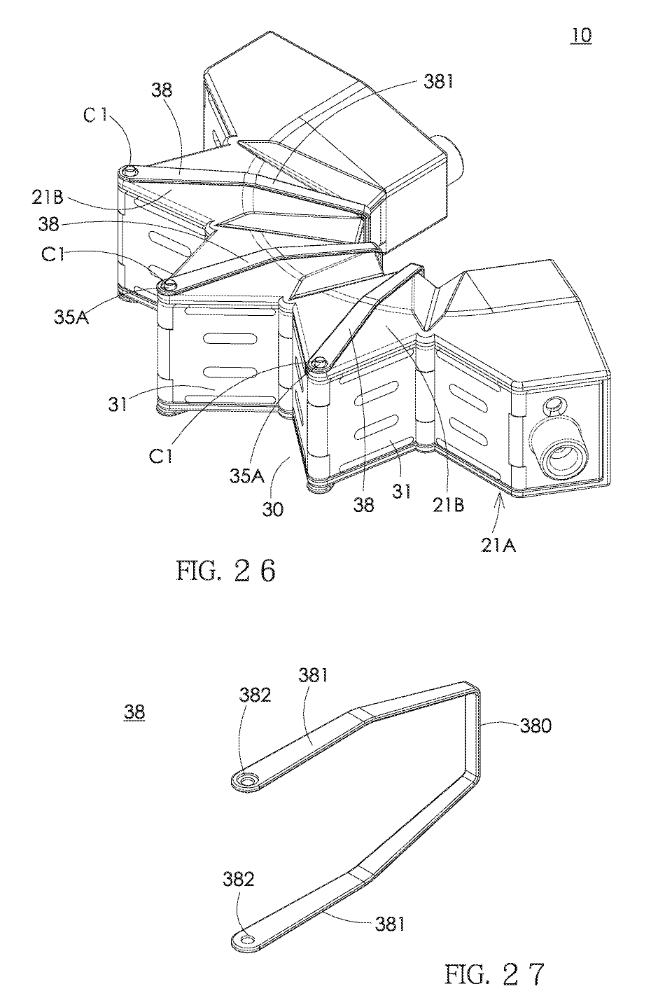

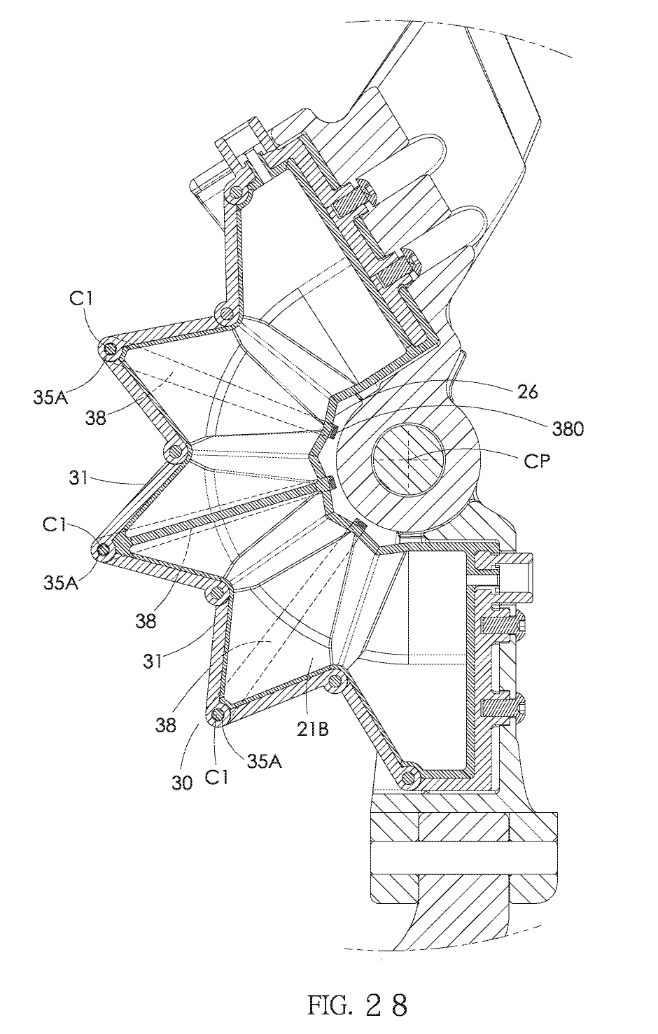

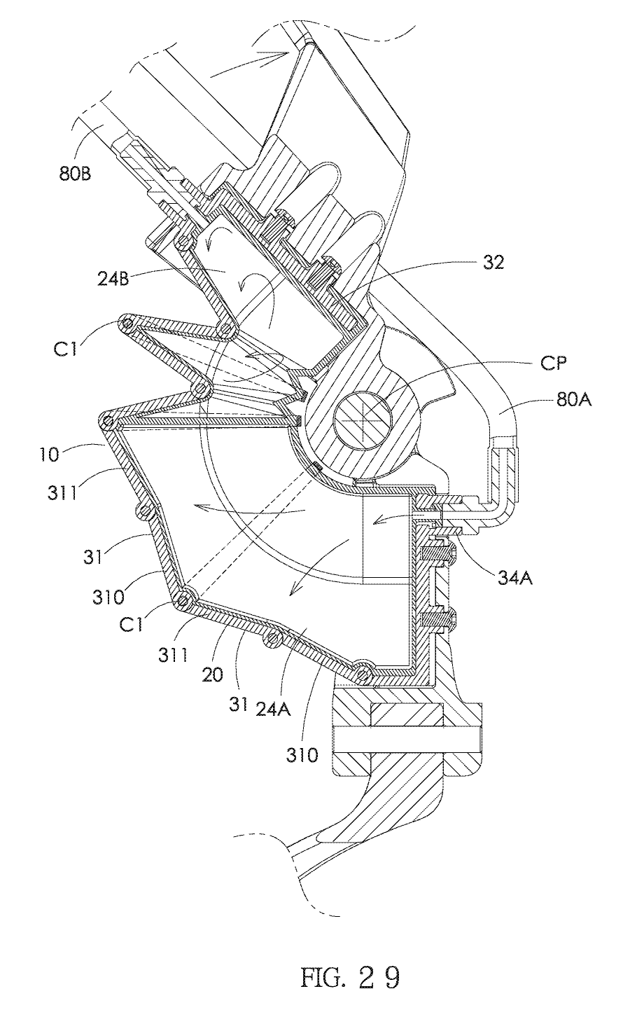

According to the above embodiment, preferably, as shown in FIGS. 26, 27 and 28, the elbow hinges 31 of the elbow hinge assembly 30 are pivotally connected to one another by a pivot rod 35A, and the pivot point of the pivot rod 35A is defined as an elbow pivot point C1. Two ends of the pivot rod 35A at the elbow pivot point C1 pass through the pair of lateral sides 21A and 21B. A U-shaped fixing bracket 38 is fixedly connected to the two ends of the pivot rod 35A. The U-shaped fixing bracket 38 includes a support rod 380 and a pair of extension portions 381 extending from two ends of the support rod 380 and each having a fixing hole 382 formed in a front end thereof and being secured to the two ends of the pivot rod 35A (by riveting), such that the pair of extension portions 381 are attached to the surfaces of the lateral sides 21A and 21B, and the support rod 380 supports the inner periphery 26 at a specific point, thereby ensuring that the distance between the elbow pivot point C1 and the center point CP remains constant. As shown in FIG. 29, when the at least one bladder chamber 24A, 24B in the bladder body 20 expands, the distance between the elbow pivot point C1 and the center point CP remains unchanged. When the knee booster 10 generates the boosting action, each elbow pivot point C1 serves as the support point at either end of each elbow hinge 31, thereby enabling the V-shaped angle between the first hinge element 310 and the second hinge element 311 of each elbow hinge 31 to expand smoothly and effectively forming a stable thrust concentrated towards the actuating link 32.

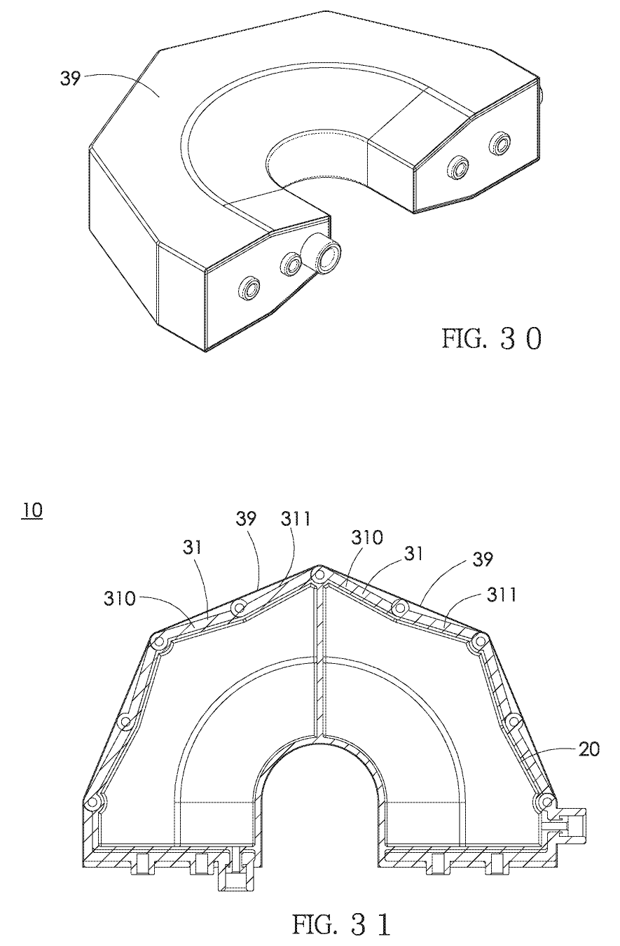

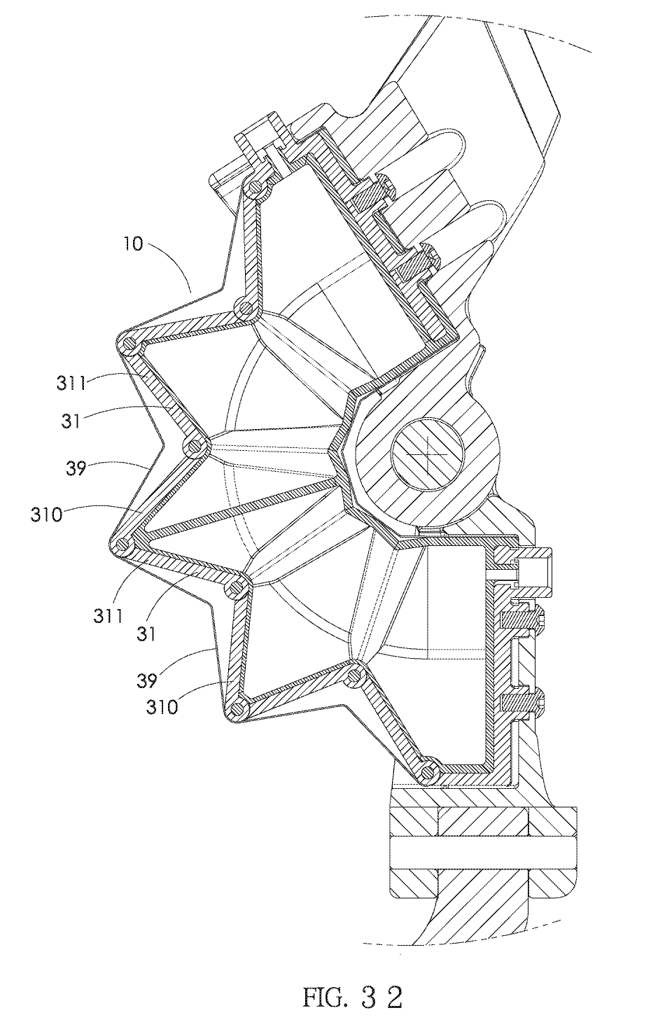

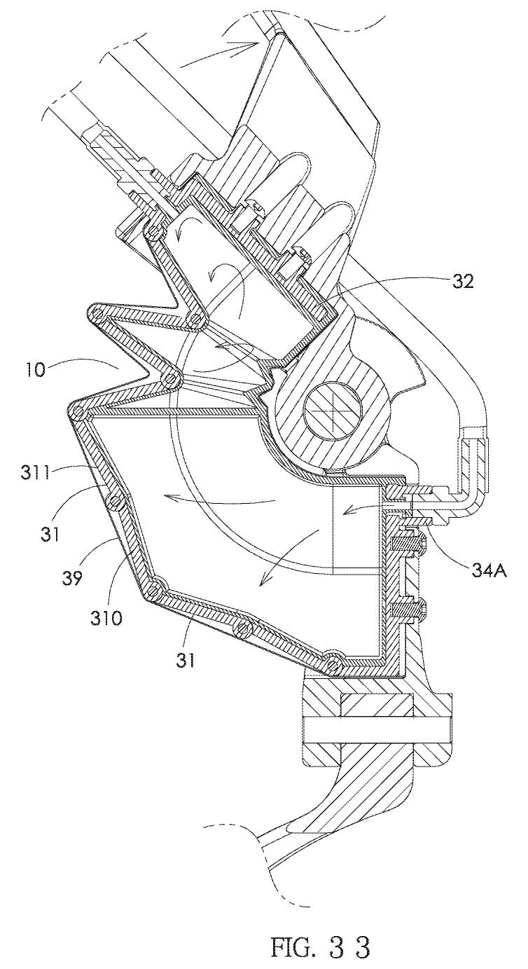

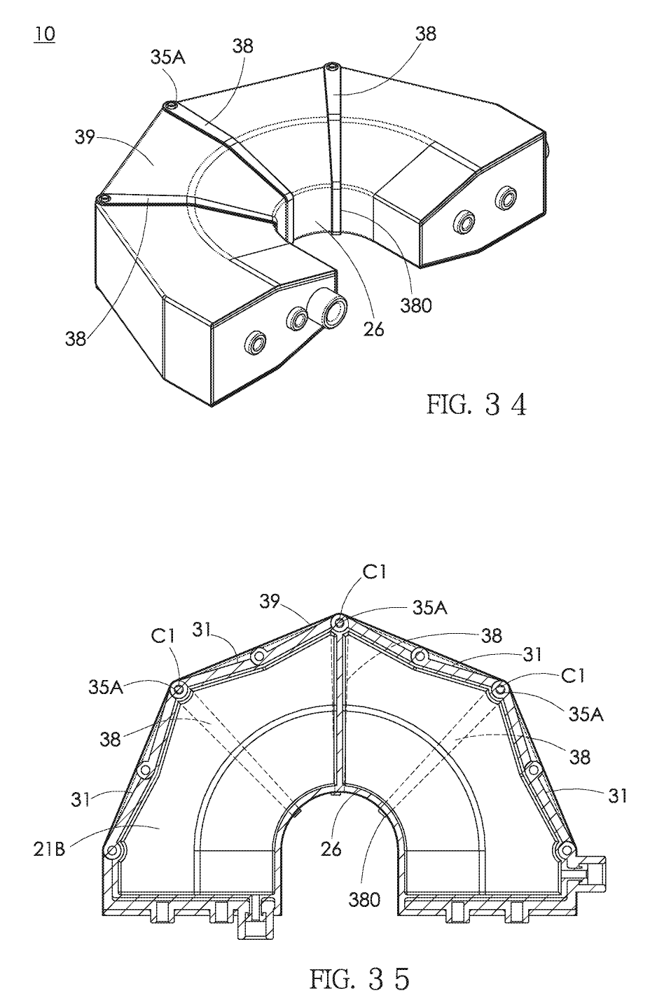

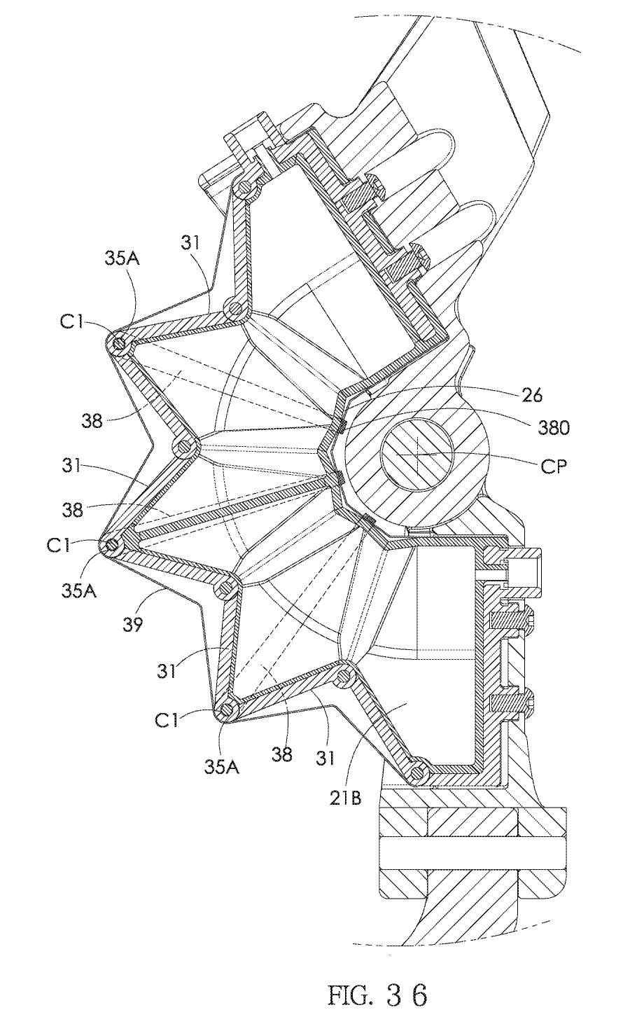

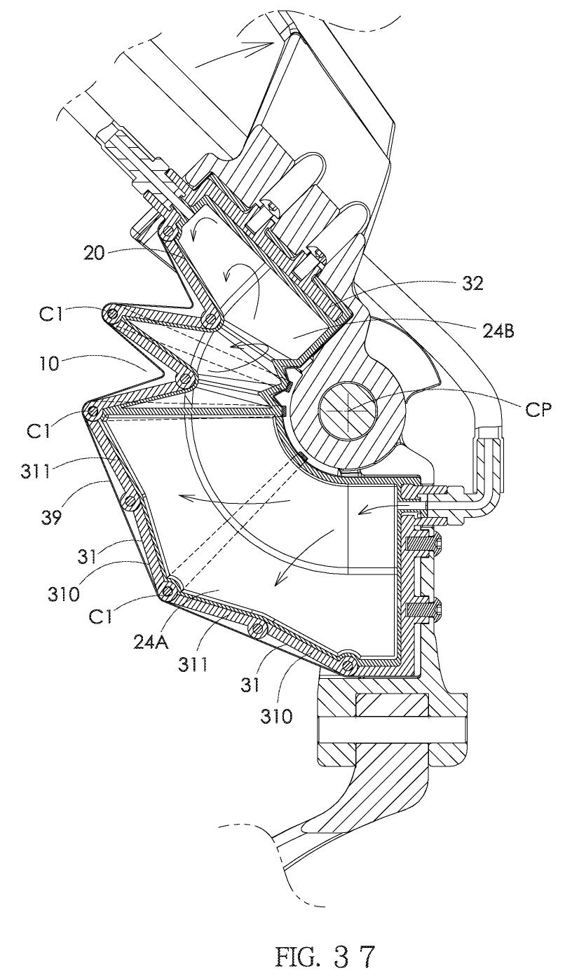

According to the above embodiment, preferably, as shown in FIGS. 30, 31 and 32, when the V-shaped angle between the first hinge element 310 and the second hinge element 311 of each of the elbow hinges 31 is close to 180 degrees, a fabric cover 39 is wrapped around the surface of the knee booster 10. The fabric cover 39 is made of a flexible and tear-resistant material (such as natural or synthetic fiber woven fabric), which can be used to restrict the shape of the bladder body 20 when it expands. As shown in FIG. 33, the fabric cover 39 restricts the V-shaped angle between the first hinge element 310 and the second hinge element 311 from exceeding 180 degrees when the first hinge element 310 and the second hinge element 311 are unfolded. When the knee booster 10 generates the boosting action, the thrust from each elbow hinge 31 can be transmitted and concentrated to the actuating link 32 stably and effectively. As shown in FIGS. 34, 35 and 36, the elbow hinges 31 of the elbow hinge assembly 30 are pivotally connected to one another by a pivot rod 35A, and the pivot point of the pivot rod 35A is defined as an elbow pivot point C1. Two ends of the pivot rod 35A at the elbow pivot point C1 pass through the pair of lateral sides 21A and 21B (referring to FIG. 7) and the fabric cover 39. A U-shaped fixing bracket 38 is fixedly connected to the two ends of the pivot rod 35A. The U-shaped fixing bracket 38, as shown in FIGS. 27, 34, 35 and 36, includes a support rod 380 and a pair of extension portions 381 extending from two ends of the support rod 380 and each having a fixing hole 382 formed in a front end thereof and being secured to the two ends of the pivot rod 35A (by riveting), such that the pair of extension portions 381 are attached to the surface of the fabric cover 39, and the support rod 380 supports the inner periphery 26 at a specific point, thereby ensuring that the distance between the elbow pivot point C1 and the center point CP remains constant. As shown in FIG. 37, when the at least one bladder chamber 24A, 24B in the bladder body 20 expands, the distance between the elbow pivot point C1 and the center point CP remains unchanged. When the knee booster 10 generates the boosting action, each elbow pivot point C1 serves as the support point at either end of each elbow hinge 31, in cooperation with the fabric cover 39 to restrict the V-shaped angle between the first hinge element 310 and the second hinge element 311 from exceeding 180 degrees, thereby enabling the elbow hinge 31 to generate a stable thrust concentrated towards the actuating link 32 effectively.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A hinged knee brace, comprising:
a knee booster, including a fan-shaped bladder body, the bladder body having a pair of lateral sides, an actuating end portion and a limiting end portion at two ends of the lateral sides, and at least one bladder chamber inside the bladder body; the bladder body being formed of an airtight material, the bladder body being in the form of an expandable, deformable flexible bladder, peripheries of the lateral sides, between the actuating end portion and the limiting end portion, being defined as an outer periphery and an inner periphery that share a common center point; the outer periphery being formed with a continuous corrugated configuration, thereby forming a plurality of deformable portions; the inner periphery being formed with folds, thereby forming a folded surface that allows for unfolding and folding; an elbow hinge assembly being disposed on the actuating end portion, the limiting end portion, and the plurality of deformable portions of the outer periphery; the elbow hinge assembly including a plurality of elbow hinges that are pivotally connected to one another and correspond in number to the plurality of deformable portions, an actuating link and a limiting link being pivotally connected to two ends of the connected elbow hinges; each of the plurality of elbow hinges including a first hinge element and a second hinge element that are pivotally connected to each other and have supporting rigidity and cooperating with a corresponding one of the deformable portions; the actuating link being pivotally connected to one end of the connected elbow hinges and fitted onto the actuating end portion and having supporting rigidity; the limiting link being pivotally connected to the other end of the connected elbow hinges and fitted onto the limiting end portion and having supporting rigidity, the bladder body having at least one connector, the at least one connector having a passage therein, the passage being in fluid communication with the at least one bladder chamber, enabling a fluid in the at least one bladder chamber to enter and exit through the at least one connector;
a thigh support, being an arm-shaped frame having supporting rigidity, the thigh support including a main arm body, a connecting portion, and a first fixing seat; the main arm body being configured for being secured to a user’s thigh with a fastening strap, the connecting portion extending from one side of a lower end of the main arm body, the first fixing seat being formed on the connecting portion; the first fixing seat being coupled with the actuating end portion of the bladder body and connected to the actuating link; a first pivot seat, centered on the center point being formed on one side of the first fixing seat;
a calf support, being in the form of an arm body with tensile strength, the calf support including a limiting arm, a support portion, and a second fixing seat; the support portion extending from a lower end of the limiting arm, the second fixing seat being disposed on an upper end of the limiting arm; the second fixing seat being coupled with the limiting end portion of the bladder body and connected to the limiting link, a second pivot seat corresponding to the first pivot seat being formed on one side of the second fixing seat, the second pivot seat being pivotally connected to the first pivot seat through a pivot.

2. The hinged knee brace as claimed in claim 1, wherein a partition is disposed inside the bladder body of the knee booster and located between the lateral sides for dividing the at least one bladder chamber into two bladder chambers, the actuating link and the limiting link each have the connector, the connector on the actuating link and the connector on the limiting link each have the passage therein, and the passages of the connectors on the actuating link and the limiting link are in fluid communication with the corresponding bladder chambers, enabling the fluid in the bladder chambers to enter and exit through the connector on the actuating link and the connector on the limiting link.

3. The hinged knee brace as claimed in claim 2, wherein the connecting portion of the thigh support has a through hole close to the first fixing seat.

4. The hinged knee brace as claimed in claim 1, wherein the elbow hinges are pivotally connected to one another, the first hinge element and the second hinge element of each of the elbow hinges are pivotally connected in a V-shape, two ends of each of the first hinge element and the second hinge element have a male hinge portion and a female hinge portion that are mated with each other and pivotally connected by a pivot rod passing through the male hinge portion and the female hinge portion, and the male hinge portion and the female hinge portion each have an engaging edge on mating end faces thereof for restricting a V-shaped angle between the first hinge element and the second hinge element of each of the elbow hinges to not exceed 180 degrees.

5. The hinged knee brace as claimed in claim 1, wherein the bladder body of the knee booster includes a box-like bladder member and a cover, and the cover and a closed side of the box-like bladder member serve as the lateral sides and seal the bladder body to form the at least one bladder chamber.

6. The hinged knee brace as claimed in claim 5, wherein a plurality of perforations and a plurality of grooves are formed on respective surfaces of the plurality of elbow hinges, the actuating link and the limiting link of the elbow hinge assembly.

7. The hinged knee brace as claimed in claim 5, wherein a plurality of recessed fold portions are formed on respective surfaces of the pair of lateral sides, and the recessed fold portions are each recessed inward toward the at least one bladder chamber.

8. The hinged knee brace as claimed in claim 1, wherein the main arm body of the thigh support has a concave surface.

9. The hinged knee brace as claimed in claim 8, wherein the main arm body has a plurality of strap holes on two sides thereof.

10. The hinged knee brace as claimed in claim 1, wherein the upper end of the limiting arm of the calf support is pivotally connected to a bottom end of the second fixing seat through a pivot shaft that is perpendicular to the pivot.

11. The hinged knee brace as claimed in claim 1, wherein the support portion formed at the lower end of the limiting arm of the calf support is an arc-shaped frame configured for resting against the dorsal side of the user’s calf, and either end of the support portion has a strap slot.

12. The hinged knee brace as claimed in claim 1, wherein the elbow hinges of the elbow hinge assembly are pivotally connected to one another by a pivot rod, a pivot point of the pivot rod is defined as an elbow pivot point, two ends of the pivot rod at the elbow pivot point pass through the pair of lateral sides, a U-shaped fixing bracket is fixedly connected to the two ends of the pivot rod; the U-shaped fixing bracket includes a support rod and a pair of extension portions extending from two ends of the support rod and each having a fixing hole formed in a front end thereof and being secured to the two ends of the pivot rod, such that the pair of extension portions are attached to respective surfaces of the pair of lateral sides, and the support rod supports the inner periphery at a specific point, thereby ensuring that a distance between the elbow pivot point and the center point remains constant.

13. The hinged knee brace as claimed in claim 1, wherein when a V-shaped angle between the first hinge element and the second hinge element of each of the elbow hinges is close to 180 degrees, a fabric cover is wrapped around a surface of the knee booster, and the fabric cover is made of a flexible and tear-resistant material.

14. The hinged knee brace as claimed in claim 13, wherein the elbow hinges of the elbow hinge assembly are pivotally connected to one another by a pivot rod, a pivot point of the pivot rod is defined as an elbow pivot point, two ends of the pivot rod at the elbow pivot point pass through the pair of lateral sides and the fabric cover, a U-shaped fixing bracket is fixedly connected to the two ends of the pivot rod; the U-shaped fixing bracket includes a support rod and a pair of extension portions extending from two ends of the support rod and each having a fixing hole formed in a front end thereof and being secured to the two ends of the pivot rod, such that the pair of extension portions are attached to a surface of the fabric cover, and the support rod supports the inner periphery at a specific point, thereby ensuring that a distance between the elbow pivot point and the center point remains constant.
